(19) United States
(12) Patent Application Publication
(10) Pub. No.: US 2025/0095832 A1
(43) Pub. Date: Mar. 20, 2025

(54) OUTREACH COMMUNICATION CONTROLS USING MACHINE LEARNING

(71) Applicant: Cerner Innovation, Inc., Kansas City, MO (US)

(72) Inventors: Rajvardhan J P, Karnataka (IN); Wasimakram Binnal, Kaenataka (IN); Pradeep Premakumar, Kaenataka (IN)

(73) Assignee: Cerner Innovation, Inc., Kansas City, MO (US)

(21) Appl. No.: 18/733,432

(22) Filed: Jun. 4, 2024

Related U.S. Application Data
(60) Provisional application No. 63/583,157, filed on Sep. 15, 2023.

(51) Int. Cl. G16H 40/20; G16H 10/60

(52) U.S. Cl. CPC G16H 40/20; G16H 10/60

(57) ABSTRACT

A system and method for predicting resource usage using machine-learning models. The method entails collecting a dataset from a variety of data sources such as electronic medical/health records or medical registries. The method identifies if an outreach communication occurred or is scheduled to occur for a subject. A set of features are extracted from the dataset and the method generates derived features from one or more extracted features. The extracted features and generated set of derived features are collated into a candidate feature vector used for training the machine-learning models. The models generate a predicted likelihood of the subject seeking care at the medical facility within a defined time period. Based on the predicted likelihoods of the subjects seeking care at the medical facility, the method predicts an upcoming resource demand at the medical facility. The method generates a recommended action in case predicted resource demand exceeds a threshold.

### CROSS-REFERENCE OF RELATED APPLICATIONS

This application claims priority to U.S. Provisional Patent Application No. 63/583,157, filed on Sep. 15, 2023, titled “OUTREACH COMMUNICATION CONTROLS USING MACHINE LEARNING,” which is incorporated by reference in its entirety for all purposes.

### BACKGROUND

The usage of resources at medical facilities can vary across facilities, departments, across days, and even across hours. For example, the number of people who are waiting to be seen (or a wait time) often varies substantially across various hospitals in the same city. As another example, the degree to which a given imaging department is on time regarding scheduled appointments may vary drastically across days.

Though medical facilities try to adapt hiring and staffing decisions based on historical and recent usage of the medical facilities, the approach is often based on human assessment of a workplace. Traditional approach uses human analysis to assess and adjust resources. Human analysis is used to predict the volume of subject visits. This frequently results in an analysis of predicting a general volume that accords with and/or matches a recent volume of observed subject visits. Department-wise resources are then allocated based on human assessment. For example, schedules may be generated for various medical providers. The allocated resources are used, such that the medical providers provide care based on the schedule (though care may be provided later than scheduled if the medical facility or part of the medical facility is over-utilized).

The individualized nature of healthcare makes it very difficult to make decisions that consistently lead to neither under-nor over-utilization of resources. Consistent under-utilization of resources can lead to substantial financial costs. Consistent (or even short-term) over-utilization of resources can result in poor subject care (e.g., due to a delay in providing the care, due to a medical provider making an error as a result of being stressed or short on time, due to not having enough of a given medication to provide to all subjects for which it would otherwise have been provided). Further, over-utilization of resources can result in medical facilities needing to prioritize various subjects (e.g., by prioritizing the availing of medication and/or physician time to subjects in critical care over others), which may lead to some under-prioritized subjects degrading and further compounding the over-utilization problem.

Thus, there is a need for new techniques that can facilitate more efficient resource usage at medical facilities.

### SUMMARY

In some embodiments, a computer-implemented method is provided to determine resource usage prediction using machine learning models. The method includes collecting an input dataset from one or more data sources for a subject and extracting a set of features from the dataset. The method generates a set of derived features from the extracted set of features. The set of extracted features and generated derived features are collated into a candidate feature vector for training a model. The method detects if an outreach communication has occurred or is scheduled to occur for a subject of a set of subjects where the outreach communication includes a recommendation that the subject seek medical care at medical facility. The method uses a trained machine learning model to predict resource usage by processing the candidate feature vector and generates a predicted likelihood of the subject seeking care at the medical facility within a defined time period. The method generates a prediction of an upcoming resource demand at the medical facility based on the predicted likelihoods of the subjects seeking care at the medical facility. The method detects if the predicted upcoming resource demand exceeds a threshold and generates an output with a recommended action related to the medical facility in response to detecting that the predicted upcoming resource demand exceeds the threshold.

In an aspect of the present disclosure, one or more data sources can be electronic health records, electronic medical records, or medical registries. The method includes extracting a set of features from the input dataset from one or more data sources. When the input data source is electronic health records or electronic medical records, extracted set of features may include demographic features (e.g. age, gender), comorbidities features (does the subject have diabetes and hyper-tension?), or anthropometric features (e.g. BMI). A registry maintains information for different clinical conditions like heart failure, diabetes, asthma etc. If a subject satisfies certain check points, then the subject information is added to the registry. A registry has different sections known as measures. These measures check the subject's condition on a regular basis. Ideally all the measures should be under pre-determined threshold. If a measure exceeds the threshold or condition is not met, that subject will be contacted from the medical facility resulting in an outreach communication.

The method may further include data preparation, data preprocessing and cleaning methods which are performed on the collected data. It may involve the transformation of data into a consistent normalized format, eliminating variations in the data representation, which makes the data suitable for analysis and comparison. Data preprocessing may also prepare, clean, and/or maintain the quality of the data and may involve tasks such as handling one or more missing values, converting a categorical set of features to a numerical set of features, correcting data anomalies and/or removing noise. The method include generating derived features by using data transformations, logical operators, statistical operations and/or criteria assessment. The method may also include a systematic process to convert each of one or more variables extracted from subject records into a candidate feature. For example, various conversions may use different absolute and/or relative thresholds to generate a set of candidate features. After data preprocessing, the extracted set of features and generated set of derived features can be collated into a candidate feature vector as training dataset which is used to train a machine-learning model and/or underlying algorithms.

The method may further include predicting, for each subject, a likelihood of the subject seeking medical care at the medical facility by processing the candidate feature vector using a machine learning model. The machine-learning model is based on learned correlations between a set of features extracted from different data sources and a set of derived features generated using the set of extracted features. The machine-learning algorithm may learn which features strongly correlate and affect the prediction of the successful outreach. The model can generate predicted usage of a resource at a medical facility as an output or can generate the predicted number of subjects visiting the medical facility within a specified period. Examples of supervised machine-learning models that can be used include the Random Forest model, Support Vector machine model, AdaBoost model, KNN, Regression, etc. The machine-learning model can also include (for example) a self-learning model, a nonlinear model, classifier sub-models, an ensemble model, and/or a client-agnostic model. By studying the various patterns of candidate feature vectors, the model generates one or more predicted outcomes as output. Based on the predicted outcomes, department-specific strategies can be developed. The strategy may (for example) indicate whether, when and/or how to change a resource allocation for a given department in view of the predicted number or when to recommend new resources for a particular department. Whether, when and/or how the resource allocation is changed in response to a given predicted number may vary across departments (e.g., given that a skill level or experience required may vary across departments).

### BRIEF DESCRIPTION OF THE DRAWINGS

The present disclosure is described in conjunction with the appended figures:

FIG. 1 is an illustrative block diagram of a computer-implemented method that may be utilized for predicting upcoming resource usage at each of one or more medical facilities using machine learning models.

FIG. 2 is a block diagram illustrating generation of prediction output from an input data collected from a data source using feature extraction and machine-learning models in accordance with some embodiments of the disclosure.

FIG. 3 is an example illustration of defining dependent variables in accordance with some embodiments of the disclosure.

FIG. 4 illustrates the flow of the present disclosure, where machine-learning model is used to predict the number of subjects, develop and implement department specific strategy and allocate the resources to departments.

FIG. 5 illustrates an exemplary process flow for tracking outreach communications and subsequent visits in accordance with some embodiments of the disclosure.

FIG. 6 illustrates the process flow of using feature vector to train an example Adaboost model to generate prediction.

FIG. 7 illustrates another example process flow of the present disclosure, where a self-learning machine learning model is used to generate the prediction.

FIG. 8 shows a dashboard for displaying insights of the recommendation, by displaying number of critical incidences, number of emergency visits and average length of stay in accordance with some embodiments of the present disclosure.

FIG. 9 illustrates an example flow of a method for obtaining the final output result of resource usage prediction using a machine-learning model trained on a set of features in accordance with some embodiments of the present disclosure.

FIG. 10 illustrates a simplified diagram of a distributed system for implementing the method of FIG. 1.

FIG. 11 illustrates a simplified block diagram of a cloud-based system environment in which various service of servers may be offered as cloud services.

FIG. 12 illustrates an example architecture of a computing system that can implement at least one example of the disclosed method.

FIG. 13 illustrates an exemplary server which may include a processor, a memory, and a mass storage device.

In the appended figures, similar components and/or features may have the same reference label. Further, various components of the same type may be distinguished by following the reference label with a dash and a second label that distinguishes among the similar components. If only the first reference label is used in the specification, the description applies to any one of the similar components having the same first reference label irrespective of the second reference label.

### DETAILED DESCRIPTION

Some embodiments of the invention relate to using a machine-learning model to determine (or predict) upcoming resource usage at one or more medical facilities (e.g., one or more medical facilities, one or more departments within a given medical facility, corresponding departments across different medical facilities, etc.). For example, a machine-learning model may be used to determine (or predict) whether a given subject will receive medical care within a predefined time period. The given subject may be one who recently received medical care (e.g., with a care visit having just ended, having been on a current day, having been within 24 hours, etc.). The given subject may alternatively or additionally be a subject on a registry floor for whom an outreach is recommended. A utilization level for each of one or more medical facilities during a future time period can then be predicted by aggregating various subject-level predictions.

The machine-learning model can be configured to receive and process a set of features derived from a sophisticated analytical approach in accordance with some embodiments of the invention. One or more of the features can include a variable that is derived from one or more underlying variables in a record associated with the subject.

The machine-learning model can be configured to predict one or more dependent variables, such as whether the subject will have a medical visit (e.g., generally, of a particular type, and/or at a particular medical facility) within a defined time window or by a defined time point. To generate labels that can be used for training, labels corresponding to the dependent variable(s) may be generated by transforming one or more related variables in a record associated with the subject.

Though the machine-learning model is trained to predict subject-level outputs (e.g., whether a given subject will have a medical visit within a defined time window), the machine-learning model may be trained using a loss function configured to assess accuracy at a medical-facility level. For example, a predicted total volume at a medical facility for a given time interval may be generated by summing a set of binary subject-specific predictions corresponding to the medical facility. In some instances, the predicted total volume may further account for a baseline volume that identifies a number of subjects who are not present based on a reason related to an issue for which they received medical care within the defined time period (e.g., 3 days, 5 days, or 7 days).

These predicted dependent variables may be used in multiple different ways. As one example, outreach to select subjects on a registry can be strategically and proactively assigned to a specific medical facility and/or the specific medical facility may be identified during the outreach as a place to receive the recommended care. As another example, a subject-specific prediction can be availed by a medical professional (e.g., via an interface) during or upon completion of an appointment or visit. Based on the subject-specific prediction, the medical professional may take various decisions for the subject such as, whether to schedule a follow-up appointment (e.g., for medical care, for a lab test, etc.), on which day to recommend for a follow-up appointment, etc. As another example, a predicted utilization level of one or more medical facilities can be used in conjunction with supply data and personnel scheduling to determine whether to transmit an alert. The alert may suggest potentially adjusting medical-provider scheduling and/or ordering more supplies (e.g., medication).

A medical registry is a systematic collection of standardized information about a group of subjects who share a condition, treatment, or other healthcare-related characteristics stored in the form of databases. These databases are often used for research, quality control, and policy decision-making. Medical registries can include information related to demographics, medical history, laboratory results, imaging data, and treatment plans. A particular medical registry may be managed by (for example) a government agency, medical facility network, research organization, insurance company, etc.

Separate medical registries may be created for each of one or more diseases, for each of one or more treatments, and/or for each of one or more subject populations. Each medical registry is associated with one or more check points that indicate which subjects are to be added to that particular medical registry. If the check points are met, the subject is added to the medical registry in a secure manner that protects data privacy.

In the context of a medical registry, “measures” refer to specific data points or metrics that are systematically collected to evaluate certain aspects of subject care, disease prevalence, treatment outcomes, or healthcare processes. These measures are selected based on their relevance to the objectives of the registry, whether that is research, quality improvement, or public health tracking. Exemplary measures include clinical measures (e.g., vital signs, laboratory results, imaging data), treatment measures (e.g., medication information, data about one or more surgeries and any complications), outcome measures (e.g., whether a subject has died, any infection, readmission to a medical facility), etc.

Sometimes, a measure is missing or is concerning. For example, each medical registry may include, for each measure, an alert condition. An alert condition may be configured to be satisfied when a given measure exceeds a threshold, when a count of given event tracked by the measure exceeds a threshold, or when a new value for the measure has not been received within a predefined amount of time, etc.

When an alert condition is satisfied, a medical professional may reach out to the corresponding subject to secure more information and/or provide a care recommendation. The care recommendation may include a recommendation to have a new medical test performed, to be seen by a medical provider, etc. Often, the recommendation is for the subject to physically go to a medical facility (e.g., for a new medical test, to receive medical care, etc.). An outreach is defined as the coordination and provision of clinical services in an outreach setting. It aims to bring primary health care directly to communities that may otherwise face barriers in secking and accessing care at fixed health center sites. In essence, outreach is an approach to meet individuals and communities where they are and to extend care in settings that fit their needs and the context of their lives. An outreach is considered to be ‘a successful outreach’ if the subject visits the recommended medical facility after the outreach for the recommended test, appointment, evaluation, etc.

While medical registries and associated outreach are very important and useful, coordinating subject outreach is complicated. Typically, the medical professional who performs outreach is a medical professional at a particular medical facility (e.g., a given medical facility), and the recommended action is for the subject to visit the particular medical facility on a same day as the outreach or within a few days. If the particular medical facility is serving a high number of subjects, adding yet another subject (or set of subjects) to be served at that time adds to over-utilization problems discussed herein. Thus, if another medical facility is under-utilized or if the subject(s) associated with the outreach reach the facility instead, even more care may be provided (e.g., reducing risks of poorer care). However, predicting how busy a given medical facility will be at a future day or time is very difficult due to the dynamic nature of subject flows. Furthermore, a time commitment for each subject may vary widely across subjects (e.g., due to varying complexities of medical conditions, varying mobility levels of subjects, varying levels of concern or questions from subjects, etc.).

In some embodiments, a machine-learning model is used to predict whether and/or when a subject who has received an outreach will participate in a medical appointment and/or receive corresponding medical treatment. To illustrate, if an outreach recommends that a subject have an appointment with a cardiologist within three days, it may be predicted whether any such appointment will occur within a time window (e.g., five days) and/or when any such appointment will occur.

### Dependent Variable Definition and Access

The machine-learning model is used to generate predictions of one or more dependent variables based on an input data set. Thus, defining such dependent variables and determining how values of such dependent variables can be accessed is important. This is important for training the machine-learning model, selecting features for the model, and evaluating the model's performance.

One or more dependent variables may indicate whether an outreach with a subject (e.g., that recommended medical care) resulted in the subject participating in a medical-care visit (e.g., within a defined time period). However, it may be useful to determine whether any such medical-care visit relates to the same medical situation that triggered the outreach. This innovation helps the medical facilities by providing a strategy which is unique to each department and using this strategy each department can operate to their full potential and provide the quality healthcare services.

### Overview

Subject care management and efficient utilization of resources is a key process for medical facilities. As it directly impacts the health of the population and the reputation of the healthcare providers. Given the increasing number of subjects each day and the ever-increasing number of diseases it caters to, a dynamic approach is needed to manage the available resources which helps the healthcare professionals to provide quality healthcare services. In traditional approaches, there may not be direct way to estimate the number of subject visits in the next few days. Hence, the medical facilities administration may not have estimate about the number of subject visits in subsequent days. This leads to medical facilities having mismatched resources, directly impacting the healthcare services. In case of high demand, the number of subject inflows can be so high that the medical facility may not have enough medicines or staff to cope with such demand.

The medical facilities may estimate the subject visits randomly by estimating subjects based on the historical data. These estimates which may be based on seasonal trends or calculated manually by the administration can be inaccurate. As the majority of these estimates utilized by many administrations are static in nature, they only focus on visible patterns without utilizing any advanced analytical solution. The medical facilities may not have an accurate estimate about the number of subject visits, and they assume the number of subject visits based on previous data which results in inaccurate estimates. Hence the medical facilities tend to overutilize or underutilize the available resources (medicines, medical facility staff etc.) which may affect the quality of medical care delivered to the subjects.

In some embodiments, techniques are provided to use machine-learning models to predict resource usage at one or more medical facilities (e.g., one or more medical facilities, one or more departments within a given medical facility, corresponding departments across different medical facilities, etc.). For example, a machine-learning model may be used to predict whether a given subject will receive medical care within a predefined period. The given subject may be one who recently received medical care (e.g., with a care visit having just ended, having been on a current day, having been within 24 hours, within 1-2 days). The given subject may alternatively or additionally be a subject on a medical registry floor for which outreach is recommended. A utilization level for each of one or more medical facilities during a future period can then be predicted by aggregating various subject-level predictions. An input dataset can be collected from a variety of data sources. The data sources include Electronic Medical Records (EMR), Electronic Health Records (EHR), or may also include medical registries. A set of features are extracted from the input dataset. The set of features may include demographic features, comorbidities features, anthropometric features etc. Features extracted from the electronic records may also include a count and/or statistics of a number of times that the subject has been admitted into a medical facility or a statistic characterizing a duration of stay in one or more medical facility admissions. In some embodiments, the method generates a set of derived features from one or more extracted features. In an exemplary scenario, ‘age’ is an extracted feature. In such scenario, the set of derived features corresponding to ‘age’ feature may include features such as max (age), min (age), mean (age), etc. that can be derived from the extracted feature ‘age’. In another exemplary scenario, using the two extracted features namely ‘emergency visit’ and ‘type of pre-conditions’, the feature ‘incident_critical’ is derived. Logical rules can be used on these two extracted features variables to derive if the subject's incident is critical or not. Derived features may also indicate an estimated total number of emergency-room visits that the subject has had within a defined time period or across the life of the subject. The method employs data pre-processing techniques and feature extraction techniques for filtering the dataset and selecting relevant features, respectively. The extracted set of features and generated derived features are collated in a candidate feature vector.

The machine-learning model is trained on the candidate feature vector associated with each subject as input variables. In an embodiment, the machine-learning model is based on learned correlations between a set of features extracted from different data sources and a set of derived features generated using the set of features. The machine-learning model may learn which features strongly correlate among each other and affect the prediction of successful outreach. The machine-learning model can predict the usage of a resource at a medical facility as an output or can generate the predicted number of subjects visiting the medical facility within a predefined period as a predicted outcome. In some embodiments, the machine-learning model is a supervised machine-learning model. Examples of the supervised machine-learning model may include the Random Forest model, Support Vector machine model, AdaBoost model, self-learning models, etc. By analyzing the various patterns in candidate feature vectors, the machine-learning model generates predictions. Based on the predicted outcomes, department-specific strategies can be developed. The strategy may (for example) indicate whether, when and/or how to change a resource allocation for a given department based on the predicted number or when to recommend new resources for a particular department. Whether, when and/or how the resource allocation is changed in response to a given predicted number may vary across departments (given that a skill level or experience required may vary across departments). As the machine-learning (ML) model keeps learning from the everyday data it also tends to improve its performance over time. Performance measures may include accuracy, precision, recall etc. When the medical facility gets an estimate about the number of subjects visits, the medical facility administration can plan for the availability of medicines, resources, essential staff, and/or other medical equipment's to deliver efficient and reliable care management.

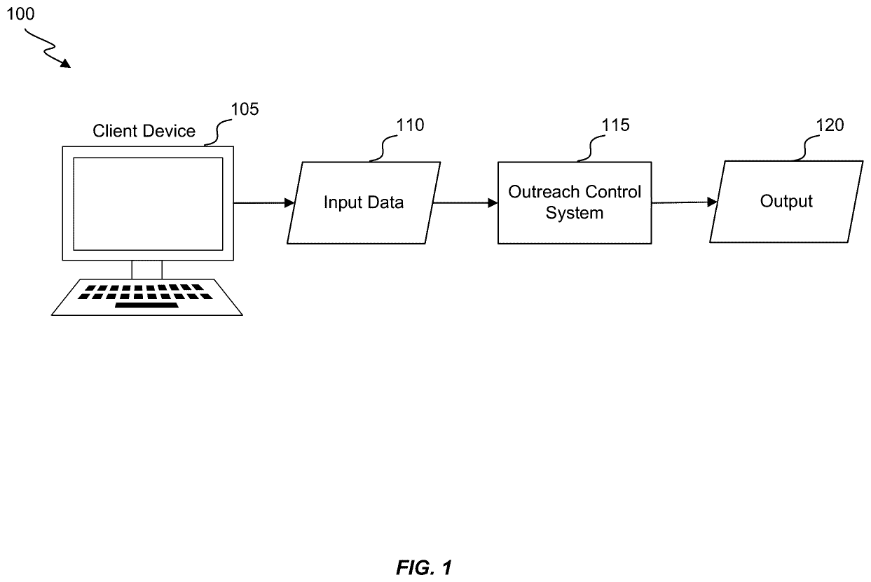

FIG. 1 is the block diagram illustrating the overview of the system that may be utilized for predicting medical facilities resource usage by predicting the likelihood of successful outreaches from input data, in accordance with an example implementation. Outreach is when a subject is contacted from the medical facility for a follow-up visit, for a test or treatment etc. An outreach is successful if the subject visits the medical facility after been contacted by the medical facility within a certain time period. A computer-implemented method 100 receives an input data 110 from a client device 105. The input data 110 is passed on to an outreach control system 115 to generate an output 120 result. The one or more computers in the client device 105 may be client terminal in communication with one or more servers, or personal digital/data assistants (PDA), laptop computers, mobile computers, internet appliances, one or two-way pagers, mobile phones, or other similar desktop, mobile or smart phones or hand-held electronic devices.

The computer system in client device 105 of the computer-implemented method 100 includes a processing system with one or more high-speed Central Processing Unit(s) (“CPU”), processors and one or more memories. The computer system in client device 105 may also include a memory for storing a plurality of processing modules or logical instructions that are executed by the one or more processors coupled. The computer memory that stores data may also be maintained on a computer readable medium including magnetic disks, optical disks, organic memory, and any other Volatile (e.g., Random Access Memory (“RAM)) or non-volatile (e.g., Read-Only Memory (“ROM), flash memory, etc.) mass storage system readable by the CPU. The computer readable medium includes cooperating or interconnected computer readable medium, which exists exclusively on the processing system or can be distributed among multiple interconnected processing systems that may be local or remote to the processing system.

Besides processor and memory, the computer system in client device 105 may also include user input and output devices such as a keyboard, mouse, stylus, and a display/touchscreen. For instance, the computer system in client device 105 may provide a means for inputting the input data 110 to memory. Input data 110 may include the dataset that can be collected from a variety of data sources. The data sources include Electronic Medical Records (EMR), Electronic Health Records (EHR), or medical registries. Electronic Medical Records are a collection of medical information about a person that is stored on a computer. An electronic medical record includes information about a subject's health history, such as diagnoses, medicines, tests, allergies, immunizations, and treatment plans. Electronic medical records can be seen by the health care providers who are taking care of a subject and can be used by them to help make recommendations about the subject's care. The data in electronic medical records may include data that is both clinical and non-clinal in nature. The EMR clinical data may be received from entities such as, but not limited to, hospitals, clinics, pharmacies, laboratories, and health information exchanges. The EMR non-clinical data may include, but not limited to, social, behavioral, lifestyle, and economic data; history, type, and nature of employment; medical insurance information; exercise information; frequency of physician or health system contact; location of residences; predictive screening health questionnaires such as the patient health questionnaire (PHQ); subject preference survey; marital status; education; housing status; and education level. The non-clinical subject data may further include data entered by subjects, such as data entered or uploaded to a social media website.

The Electronic Health Records (EHRs) are systematized collection of subjects and population electronically stored health information in a digital format. These records can be shared across different health care settings. Records are shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information. Features in electronic health records may include demographic features (e.g. age, gender), comorbidities features (does the subject have diabetes and hyper-tension?), or anthropometric features (e.g. BMI). An EMR captures information from a single care provider, which is only available to that one care provider. However, EHRs are designed to be used by multiple care providers and healthcare organizations. Features extracted from the electronic records may also include a count or statistics as to a number of times that the subject has been admitted into a medical facility or a statistic characterizing a length of stay of one or more medical facility admissions. Each medical facility take care of different registries based on the infrastructure availability. A medical registry maintains information for different clinical condition like heart failure, diabetes, asthma etc. If a subject satisfies certain check points, then the subject information is added to the registry. A registry has different sections known as measures. These measures check the subject's condition on a regular basis. Ideally all the measures should be under pre-determined threshold. If a measure exceeds the threshold or condition is not met, that subject will be contacted from the medical facility resulting in an outreach communication.

The input data may also receive data from health information exchanges (HIE). HIEs are organizations that mobilize healthcare information electronically across groups within a region, community or different medical facilities. HIEs are developed to share clinical and non-clinical subject data between healthcare entities within cities, states, regions or within health systems.

The outreach control system 115 processes the data received from the input data 110 and generates the output 120 result comprising of medical facilities resource usage by predicting the likelihood of successful outreaches within a certain time period.

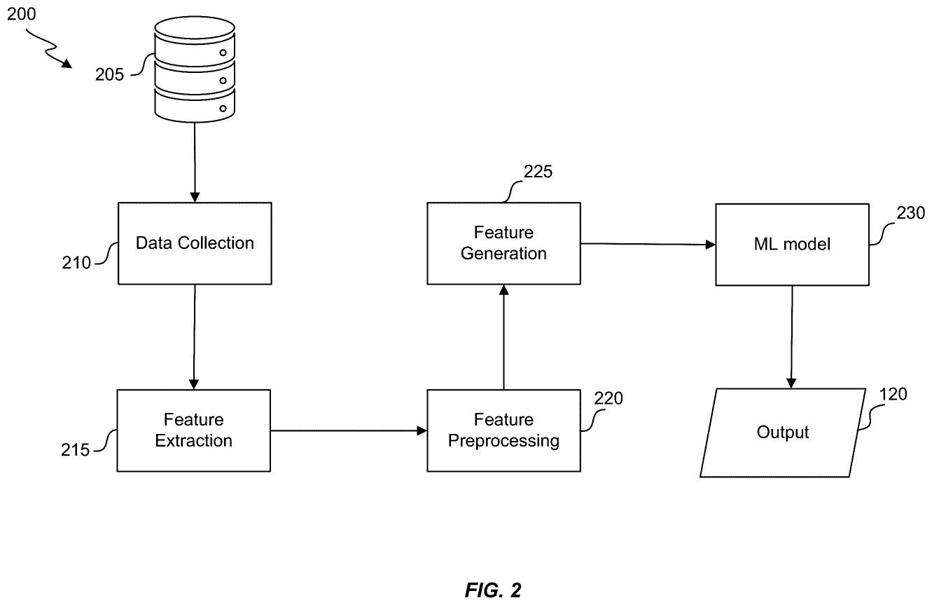

FIG. 2 is a block diagram that illustrates a process 200 for predicting output corresponding to input data collected from a data source by way of feature extraction technique and machine-learning models, in accordance with an exemplary embodiment of the present disclosure.

At block 205, subject data is stored and/or organized at one or more data sources. The subject data may represent one or more recent and/or current (within a defined number of days) encounters, visits, appointments, etc. with one or more subjects. The data source may include (for example) a computing system (that includes one or more cloud-computing components) associated with a medical facility (e.g., medical facility network, medical facility, medical facility department, urgent care, physician's office, medical laboratory, etc.). The subject data may be stored and/or organized in accordance with one or more predefined structures and/or formats. For example, the subject data may be stored and/or organized in accordance with an Electronic Medical Record format or a Health Information Exchange format. Such data may be stored (for example) in accordance with defined key-value structure, in one or more tables or arrays, in log-data format, etc.

At block 210, the subject data is collected from the one or more data sources. The data collection can be performed in a manner to ensure that a requesting system is authorized to access the subject data from the data sources. In some instances, the subject data is processed to introduce anonymization and/or to obscure personally identifiable information to secure subject's privacy. Data collection 210 may be used to facilitate the transfer of the subject data from the data sources to the subsequent stages of a data wrangling process. This can help in maintaining the integrity and consistency of the subject data. Data collection combines data from different data sources, using unique identifiers to avoid discrepancies, and utilizing gender and age as demographic identifiers, thus enabling the process to exploit diverse information. The Data collection extracts clinical and non-clinical data from the data sources in real-time or in batch files using medical facilities accepted protocols. The Data collection process may include Extract, Transform, and Load (ETL) processes for collecting subject data from different sources, transforming the data into a standardized format, and generating a unified input dataset. The process ensures data quality and consistency, tracks historical data, provides a unified view of data from various sources, simplifies data integration, and facilitates prediction analysis.

Feature extraction 215 is used to extract dependent features from the input dataset. The dependent features may relate to variables that influence utilization of a medical facility. In some instances, a dependent feature further relates to whether and/or how a subject responded to an outreach communication, proactive scheduling of a follow-up appointment, etc., to indicate whether, when and/or where the subject participated in a corresponding follow-up appointment or visit. A feature may also indicate an estimated total number of emergency-room visits that the subject has had within a defined time period or across the life of the subject. Dependent variables are further discussed herein (e.g., see the Dependent Variable Definition and Access section).

At block 220, data preparation and data filtering are performed on the data collected at block 215. It involves the transformation of data into a consistent normalized format, eliminating variations in the data representation, which makes the data suitable for analysis and comparison. Normalized data can be generated using various normalization techniques and algorithms. It may include z-score normalization which normalizes each data point using mean and standard deviation, linear normalization which normalizes each data point using minimum and maximum feature values present in the data, and standard deviation normalization which uses standard deviation to normalize each data point, etc. This step transforms the subject data in various formats into standardized data.

After normalization, the data may be passed through a series of data wrangling steps such as data parsing, data conversion, and data transformation to extract the relevant data that can be used for training the models. Feature preprocessing 220 can include the methods to prepare, clean, and maintain the quality of the data received from block 215. Feature preprocessing involves tasks like handling one or more missing values, converting a categorical set of features to a numerical set of features, correcting data anomalies, and removing noise. The one or more missing values can be handled by using techniques such as imputation and data removal. Missing value of a field may (for example) be replaced with a default value for a field (e.g., that is determined based on a statistical analysis of other corresponding values for the same field). The default value may be a mean value of the values of a field when the field's value is numeric. When the missing value is categorical, the default value may be a median or mode value of the values of the field. The conversion of the set of features that have categorical value to the set of features with numerical value can be achieved using various techniques and algorithms such as One-Hot Encoding, Label Encoding, Binary Encoding, Helmert Encoding, etc. Subject data acquired from various data sources may include noise which refers to random or unpredictable fluctuations in data that disrupt the ability to identify target patterns or relationships. Statistical methods such as mean, median, and quantiles can be used to detect anomalies and noise in the input dataset to generate clean data. In addition, data visualization and exploratory data analysis techniques can also be used to detect anomalies and remove noise in the dataset. The clean data can then be used to train machine-learning models and/or used by underlying algorithms. The preparation and preprocessing may also address data that has a data type inconsistent with that from another source/time and/or that is to be received by the machine-learning model. The data preparation and preprocessing may include extracting and/or identifying variables from the subject data that are to be used for feature generation and/or training of the machine-learning model.

Feature generation 225 is used to extract features from the cleaned and preprocessed input dataset and generate derived features. The features can be generated and/or selected in accordance with the disclosure provided hereinbelow.

### Feature Generation and Selection

The dimensionality, size and complexity of medical records is very high. Further, many variables are descriptive and/or are real numbers. Meanwhile, for a machine-learning model to be well-trained, the size of a training data set must be sufficiently large to sample various combinations of potential input variables. Thus, the size of the training data required to train a machine-learning model typically exponentially scales as more variables are included in an input data set and as the entropy of each input variable is higher.

In some embodiments of the invention, derived features can be generated using data transformations, logical operators, statistical operations and/or criteria assessment. Such an approach may reduce the cardinality of a variable and/or the dimensionality of a feature space. For example, text data from the record(s) may be converted to a numeric feature by using natural language processing, semantic mapping, keyword detection, etc. As another example, categorical data from the record(s) may be converted to a numeric feature. As another example, a numerical value may be transformed into a binary value or categorical value (e.g., representing a range assignment) using comparisons with one or more thresholds. As yet another example, a counter may be used (e.g., potentially with natural language processing, semantic mapping, keyword detection, etc.) to count a number of occurrences of representations of a given type of event.

In some instances, a systematic process is used to convert one or more variables extracted from subject records into a derived feature which may be stored in the candidate feature vector. For example, various conversions may use different absolute and/or relative thresholds (e.g., to arrive at a greater/less-than result, to arrive at a range assignment, etc.) to generate a set of candidate features. As another (additional or alternative) example, various logical operands and/or data-variable combinations may be used to generate a set of candidate features. Thus, multiple candidate features may be generated that include an assessment of the same underlying variable (e.g., where none, some or all of the multiple candidate features may, or may not, be based on an assessment of another underlying variable). For instance, the variable “incident_critical” is derived using the “type of pre-conditions” and “emergency visit” of the subject. Logical rules can be used on the two extracted features to derive the information regarding the subject's incident if it is critical or not. As an additional illustration, the new derived features that may be obtained from the extracted feature “age” include max (age), min (age), mean (age), etc. Derived features may also include an estimate of the total number of emergency-room visits that the subject has had throughout the course of their lifetime or within a specified time frame. One or more candidate features may be selected from the set of extracted features and the set of derived features.

The candidate features may be evaluated individually or collectively to generate a candidate-feature score for each candidate feature. The evaluation may be configured to estimate the extent to which each candidate feature is predictive of one or more dependent variables for training the machine-learning models. In some instances, the evaluation accounts for an extent to which various combination of the variables (or transformed representations thereof) are synergistic. Such that the combination provides more information about the dependent variable(s) relative to a sum of the information that would be provided by each of variables individually. Similarly, in some instances, the evaluation accounts for whether and/or an extent to which various combination of variables (or transformed representations thereof) are redundant. Such that the combination provides less information about the dependent variable(s) relative to a sum of the information that would be provided by each of the underlying variables individually.

The evaluation of the candidate-feature scores can include using a statistical test, function fitting, or another approach. For example, a bivariate test (e.g., an ANOVA test) that predicts the extent to which various candidate features contribute to an accurate prediction of the dependent variable(s) can be used. Such a test may further include a statistical confidence of each prediction. As another example, an information-theory analysis may be used to quantify the degree to which each of one or more candidate features reduced an uncertainty about the predicted dependent variable. Further or alternatively, an information-theory analysis may identify redundancy and synergy across one or more candidate features.

In some instances, the candidate-feature scores are used to iteratively fine-tune a process of converting input dataset variables into derived features. For example, one or more thresholds may be iteratively adjusted based on corresponding scores. As another example, if a feature relies on multiple variables, a weight applied to the variables can be iteratively adjusted.

A feature-defining criterion may be defined to cease evaluation of candidate features and to define features to use to generate predictions that will be output and/or used. The feature-defining criterion may be defined to include a convergence threshold, a performance threshold (e.g., of a predicted accuracy based on test or validation data), etc. In some instances, the feature-defining criterion further includes a timing constraint to indicate that a top set of candidate features is to be selected by a given time based on the performance threshold.

When the feature-defining criterion is satisfied, a subset of the candidate features is selected based on the evaluation. The subset of the candidate features may include each candidate feature associated with a candidate-feature score that is above a relative or absolute threshold. The subset may include each candidate feature that is represented in a candidate set of candidate features, where the candidate set has features based on performance evaluation. The subset of the candidate features is used to generate the input data for the machine-learning model, that may change over time. The feature evaluation and/or selection may be repeatedly performed to dynamically adjust the selected candidate feature set. As one example, such an evaluation and/or selection may be performed daily. This approach may facilitate adjusting to seasonal shifts, temporal trends, etc. The subset of candidate features are collated into a candidate feature vector which may be used to train a machine-learning model and/or used by the underlying algorithms. Augmented with such derived features, the analysis and predictive modeling performed by the present system to identify the subjects visiting a particular medical facility become much more robust and accurate.

The candidate features and/or the selected subset of candidate features can include one or more features as presented in Table 1.

TABLE 1
Candidate features list

| S No | Feature Name | Description |
|---|---|---|
| 1 | Incident_critical | A derived variable-based classification |
| | | of type of criticality based into 4 levels |
| | | 0, 1, 2 &3 which is converted to 0 and 1 |
| | | before feeding to model. The logic for |
| | | this is based on how to identify the |
| | | critical subjects among all. For |
| | | example, one subject might have |
| | | history of highest emergency visits |
| | | though for present he might not show |
| | | emergency but due to historical data |
| | | would be classified as partly |
| | | incident_critical. |
| 2 | emergency_visits | The total number of emergency visits a |
| | | subject has had across medical |
| | | facilities. This number will be between |
| | | 0 to infinity. |
| 3 | type_of_preconditons_all | Total number of preconditions and |
| | | allergies all records indicate that a |
| | | given subject has. This variable is |
| | | converted to 0 and 1 before feeding to |
| | | model. |
| 4 | comorbities_influence | Total comorbidities a subject has value |
| | | ranges from 0 to max. |
| 5 | Critical_insubjects_avg | The average number of times a subject |
| | | was admitted more than once in last 3 |
| | | years. The value can range from 0 to |
| | | max. |
| 6 | length_stay_admission | Total number of days admitted in last 3 |
| | | years. Ranges from 0 to max. |
| 7 | length_stay_admission_avg_all | Is total number of days admitted in last |
| | | 3 years > average 3 years admission |
| | | for all subjects. Ranges between 0 and 1. |
| 8 | length_stay_critical | Length of stay for critical illness in last |
| | | 3 years. Ranges from 0 to max. Critical |
| | | illness is taken from encounters |
| | | categorized as emergency and |
| | | endangering class. |
| 9 | length_stay_critical_avg | Is total number of days admitted for |
| | | critical illness in last 3 years > average |
| | | 3 years admission for all critical |
| | | subjects. Ranges between 0 and 1. |
| 10 | Department_dependency | Total number of inter-department |
| | | referrals for subject in last 3 years. |
| | | Ranges from 0 to max |
| 11 | Department_dependency_avg_comorbities | Total number of inter-department |
| | | referrals for subject in last 3 years/by |
| | | total number of comorbidities. Ranges |
| | | from 0 to max. |
| 12 | Incident_critical_percentile | The most critical emergencies of a |
| | | subject. Total number of incidents |
| | | critical in last 3 years. A scale for all |
| | | subjects who lie between 90th |
| | | percentile and 100th percentile. Ranges |
| | | from 0 to max. |
| 13 | Incident_critical_percentile_length_stay | Incident_critical_percentile total |
| | | length of stay in last 3 years. Ranges |
| | | from 0 to max. |
| 14 | Inpatient_length_stay_vs_interdepartment | Total length of stay as admission/by |
| | | the number of departmental changes. |
| | | Ranges from 0 to max. |
| 15 | Inpatient_length_stay_vs_interdepartment_critical | Inpatient_length_stay_vs_interdepartment - |
| | | linked to either cancer, heart |
| | | attack, accidental, unconscious |
| | | encounters. Range is 0 to 1. |
| 16 | Subject_visit_noadmissions | Total visits which didn't have |
| | | admissions in last 3 years. Ranges |
| | | from 0 to max. |
| 17 | subject_visits_interdepartment_high | Total visits which didn't have |
| | | admissions in last 3 years between |
| | | 90th to 100th percentile. Ranges from |
| | | 0 to max. |
| 18 | Subject_visit_interdepartment_vs_comorbities | Total visits which didn't have |
| | | admissions in last 3 years between |
| | | 90th to 100th percentile/by total |
| | | number of comorbidities. Ranges from |
| | | 0 to max. |
| 19 | Subject_visits_regular | Number of subjects visiting for |
| | | noninvasive checkups after discharge |
| | | in last 3 years. Ranges from 0 to max. |
| 20 | Subject_visits_regularvsnon_regular | Subjects who fall into regular subjects |
| | | from Subject_visits_regular will be 1. |
| | | Range from 0 to 1. |
| 21 | subject_visits_regular_critical | Total visits in last 3 years for checkups |
| | | but have history of critical illness like |
| | | cancer, cardiac illness, had risky |
| | | accident in history. Range 0 to 1. |
| 22 | subject_visit_critical_age | Total number of subjects suffering |
| | | from critical illness above the age of |
| | | 55 years. Range from 0 to max. |
| 23 | subject_visit_age_comorbities | Number of subjects over the age of 55 |
| | | years/by total number of |
| | | comorbidities. Range from 0 to max. |
| 24 | MAX(numeric_df.admission_source_raw_code) | |
| 25 | MAX(numeric_df.admission_type_raw_code) | |
| 26 | MAX(numeric_df.age) | |
| 27 | MAX(numeric_df.comm_serial_no) | |
| 28 | MAX(numeric_df.discharge_disposition_code) | |
| 29 | MAX(numeric_df.discharge_disposition_raw_code) | |
| 30 | MAX(numeric_df.encounter_number) | |
| 31 | MAX(numeric_df.financial_class_code) | |
| 32 | MAX(numeric_df.financial_class_raw_code) | |
| 33 | MAX(numeric_df.service_provider_npi) | |
| 34 | MEAN(numeric_df.admission_source_raw_code) | |
| 35 | MEAN(numeric_df.admission_type_raw_code) | |
| 36 | MEAN(numeric_df.age) | |
| 37 | MEAN(numeric_df.comm_serial_no) | |
| 38 | MEAN(numeric_df.discharge_disposition_code) | |
| 39 | MEAN(numeric_df.discharge_disposition_raw_code) | |
| 40 | MEAN(numeric_df.encounter_number) | |
| 41 | MEAN(numeric_df.financial_class_code) | |
| 42 | MEAN(numeric_df.financial_class_raw_code) | |
| 43 | MEAN(numeric_df.service_provider_npi) | |
| 44 | MIN(numeric_df.admission_source_raw_code) | |
| 45 | MIN(numeric_df.admission_type_raw_code) | |
| 46 | MIN(numeric_df.age) | |
| 47 | MIN(numeric_df.comm_serial_no) | |
| 48 | MIN(numeric_df.discharge_disposition_code) | |
| 49 | MIN(numeric_df.discharge_disposition_raw_code) | |
| 50 | MIN(numeric_df.encounter_number) | |
| 51 | MIN(numeric_df.financial_class_code) | |
| 52 | MIN(numeric_df.financial_class_raw_code) | |
| 53 | MIN(numeric_df.service_provider_npi) | |
| 54 | SUM(numeric_df.admission_source_raw_code) | |
| 55 | SUM(numeric_df.admission_type_raw_code) | |
| 56 | SUM(numeric_df.age) | |
| 57 | SUM(numeric_df.comm_serial_no) | |
| 58 | SUM(numeric_df.discharge_disposition_code) | |
| 59 | SUM(numeric_df.discharge_disposition_raw_code) | |
| 60 | SUM(numeric_df.encounter_number) | |
| 61 | SUM(numeric_df.financial_class_code) | |
| 62 | SUM(numeric_df.financial_class_raw_code) | |
| 63 | SUM(numeric_df.service_provider_npi) | |
| 64 | MAX(categorical_df.admission_source_code) | |
| 65 | MAX(categorical_df.admission_source_display) | |
| 66 | MAX(categorical_df.admission_source_primary_display) | |
| 67 | MAX(categorical_df.admission_type_code) | |
| 68 | MAX(categorical_df.admission_type_display) | |
| 69 | MAX(categorical_df.admission_type_primary_display) | |
| 70 | MAX(categorical_df.classification_code) | |
| 71 | MAX(categorical_df.classification_display) | |
| 72 | MAX(categorical_df.classification_primary_display) | |
| 73 | MAX(categorical_df.classification_raw_code) | |

ML model 230 receives the candidate feature vectors from feature generation 225 and includes a machine-learning model that enables the prediction of the resource usage. The machine-learning model is trained on the set of extracted and derived features associated with each subject as input variables. In an embodiment, the machine-learning model is based on learned correlations between a set of features extracted from different data sources and a set of derived features generated using the set of features. The machine-learning model may learn which features correlate and affect the prediction of the successful outreach. The machine-learning model can predict usage of a resource at a medical facility as an output or can generate the predicted number of subjects visiting the medical facility within a specified period. Any supervised machine-learning model can be deployed. Examples of supervised machine-learning models that can be used include the Random Forest model, Support Vector machine model, AdaBoost model, KNN, Regression, etc. By studying the various patterns of candidate feature vectors, the model generates predictions. A machine-learning model can be trained using the derived features and the extracted features and generates one or more predicted outcomes as output 120.

The machine-learning model can also include (for example) a self-learning model, a nonlinear model, classifier sub-models, an ensemble model, and/or a client-agnostic model. The machine-learning model may use one or more decision-tree sub-models. For example, the machine-learning model may be an ensemble technique that is based on multiple decision-tree analyses. Such an ensemble technique may incorporate a boosting approach, such as Adaptive Boosting (Adaboost). During a development and testing phase, the machine-learning model may be trained on a portion of a training data set and validated using a remaining portion (e.g., using a 70:30 split). Model performance can be evaluated using the root mean squared error (RMSE) and/or the mean absolute percentage error (MAPE) metrics.

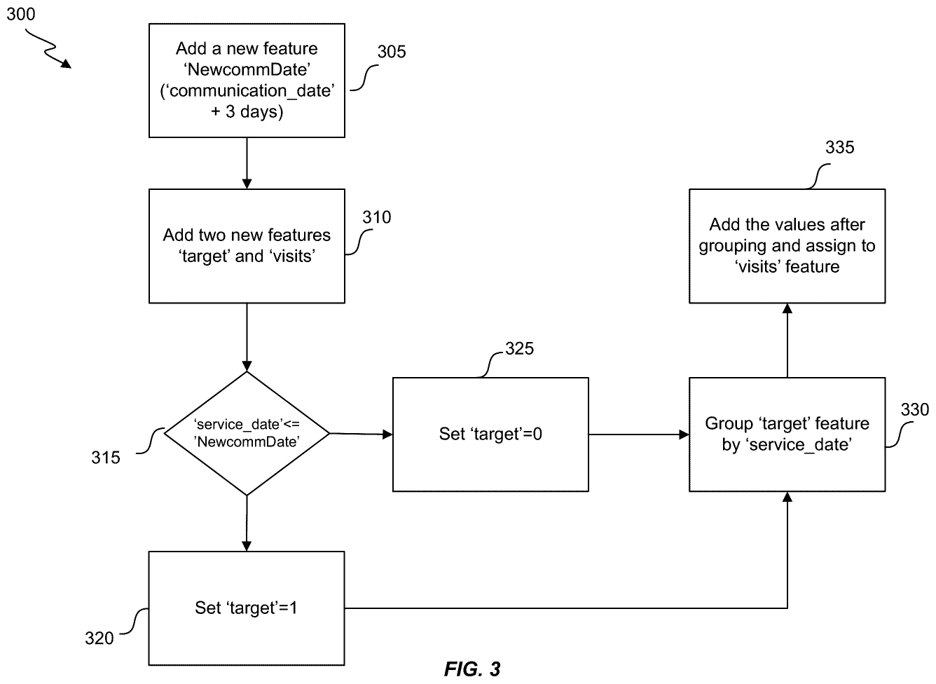

FIG. 3 illustrates a flow chart depicting a process 300 for defining dependent variables, according to an exemplary embodiment. At block 305, for each subject that received an outreach, a threshold date for receiving care in accordance with an outreach recommendation is determined. The threshold date may be defined as a predefined number of days after the outreach occurred or a specific date indicated in the outreach. A new feature can be defined for each subject that received outreach, and added to a data structure where the new feature identifies the target date.

To retrieve such information regarding the outreach communication, during storage of outreach data, a unique identifier may be associated with each outreach. For example, the unique identifier may be generated based on an identifier of the subject and a date (and/or time) of the outreach.

At block 310, for each subject, subject data is accessed from 205 and added to a data structure that indicates any medical appointment or visit that the subject had within a defined period and the type of medical appointment or visit.

At block 315, it is determined if there was any medical appointment or visit—that occurred by the threshold date. When the medical appointment or visit occurred by the threshold date, “target” variable is set to 1 at block 320, otherwise it is set to 0 at block 325. It is thus determined whether any medical appointment or visit is accorded with the recommended “target” medical care indicated during the outreach.

In some instances, multiple medical appointments and/or visits may have occurred, and they may be the same and/or different in terms of whether they occurred by the threshold date and/or whether they accorded with the “target” medical care of the outreach. Thus, at block 330, when multiple medical appointments or visits are detected that correspond to the “target” medical care by the threshold date, they are grouped together. Such groupings may preserve information about when such appointments or visits occurred relative to each other and/or relative to the outreach.

Additionally, or alternatively, “target” medical care may be defined as care that is received by a same department or type of department associated with a condition, treatment, etc. of a registry that prompted the outreach. Thus, information about an outreach, a subject, and one or more medical-care encounters can be used to estimate a number of medical-care encounters that occurred with respect to the subject in response to recommendations provided in the outreach.

To illustrate, a first dependent variable (i.e., a ‘target’ variable) may be a binary variable that can either have a value of ‘0’ or ‘1’. In an exemplary scenario, when a subject visits a prescribed medical facility within a recommended time period, the value of the first dependent variable is set to ‘1’. Else when the subject does not visit the prescribed medical facility, the value of the first dependent variable is set to ‘0’. A second dependent variable (i.e., a ‘visits’ variable) indicates a total number of subjects that visited the prescribed medical facility on a particular day. The second dependent variable can be calculated by determining a sum of ‘target’ variables on a particular day using the reference feature ‘service date’ resulting in a numeric feature.

At block 335, grouped values of the variables are added to a data structure. Further, one or more dependent-variable labels can then be generated based on the visit information (e.g., that indicates whether a follow-up medical-care visit occurred, when it occurred and/or whether it was of a recommended type of medical-care visit).

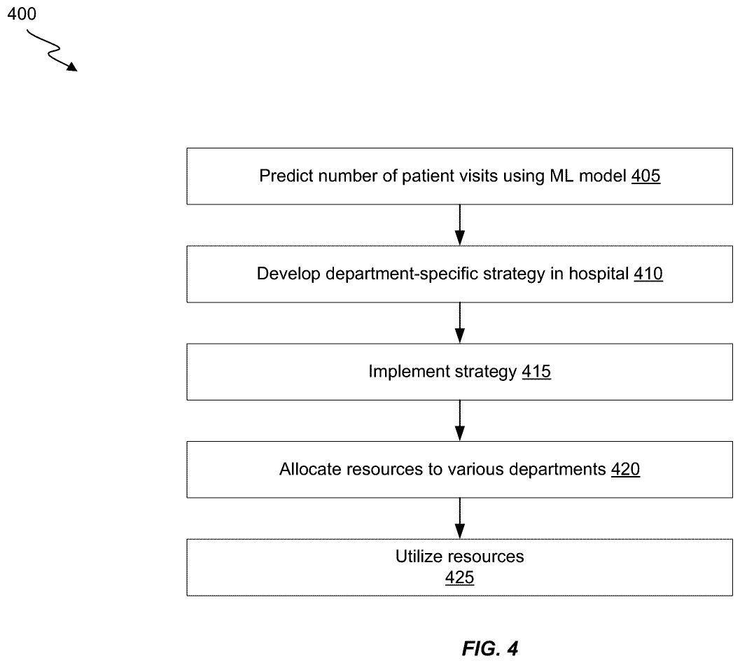

FIG. 4 illustrates an exemplary process flow 400 for allocating resources using a machine-learning model, in accordance with some embodiments of the invention.

At block 405, a number of subject visits is predicted using a machine-learning model. The predicted number of subject visits may indicate how many subject visits are predicted to occur within a specified time period (e.g., the next three days, the next five days, the next ten days, etc.). In some instances, the predicted number of subject visits corresponds to visits of subjects occurring in response to an outreach that has occurred (and/or, in some instances, is scheduled to occur). The prediction may correspond to a number of visits across a medical facility network, a number across medical facilities in a given medical facility network and within a given region, a number across a given type of department in medical facilities, a number for an individual medical facility, or a number for an individual department in an individual medical facility.

In some embodiments, the machine-learning model can be an Adaboost model. In some aspects of the present disclosure, the machine-learning model may have properties of at least one of, a self-learning Artificial Intelligence (AI) model, non-linear AI model, AI based classifier sub-model, AI based client agnostic, etc.).

At block 410, a department-specific strategy is developed. The strategy may indicate whether, when and/or how to change a resource allocation for a given department in view of the predicted number of subjects visits. For example, when the predicted number is relatively higher than the average number of visits, a computer interface may transmit or display a recommendation that a new supply or medication resources is to be purchased, and/or more human resources should be recruited (e.g., by reassigning a medical professional from one department to another at least temporarily, by recruiting a contract medical professional, and/or by requesting that a medical professional in the department work overtime, etc.). The resource allocation may change in response to a given predicted number and may vary across departments (e.g., given that the skill level or experience required may vary across departments).

This process involves clubbing an outcome of the machine-learning model with a strategy. The strategy is unique for each department (for example the emergency department would have high priority resources allocation along with proactive report generation at each stage while the ENT departments would have moderate priority). Due to adaptive learning and continuous training on new data generated daily, performance of the machine-learning model enhances with time. When the medical facility gets an estimate about the number of subjects visits, the facility can plan for the availability of medicines, resources, essential staff, and other medical equipment's to deliver proper care management.

At block 415, upon receiving a signal from a system authorized to determine resource allocation that confirms that the department-specific strategy (or a specified modification thereof) is to be implemented, the strategy is implanted. Such implementation may include initiating or completing a purchase order for medications or supplies, updating a schedule for human resources and distributing the updated schedule, generating and transmitting a request to a device of one or more human resources to request overtime commitment, generating and transmitting a request to a device associated with one or more contract medical professionals that requests assistance, etc. The strategy may include an iterative approach of securing additional resources (human or supplies) based on the responses to various requests. The strategy may also include providing recommendations to the subject to visit a different medical facility to reduce the load of a particular medical facility. The strategy may also include generating an updated schedule that assigns resources to time slots associated with the particular medical facility. The updated schedule may include adding a new resource to one or more time slots associated with the particular medical facility or proposes extending at least one time slot currently assigned to a given resource.

At block 420, resources are allocated to various departments by a variety of steps such as by updating a schedule, transmitting a notification confirming new resources, etc. At block 425, the resources are used at the various departments.

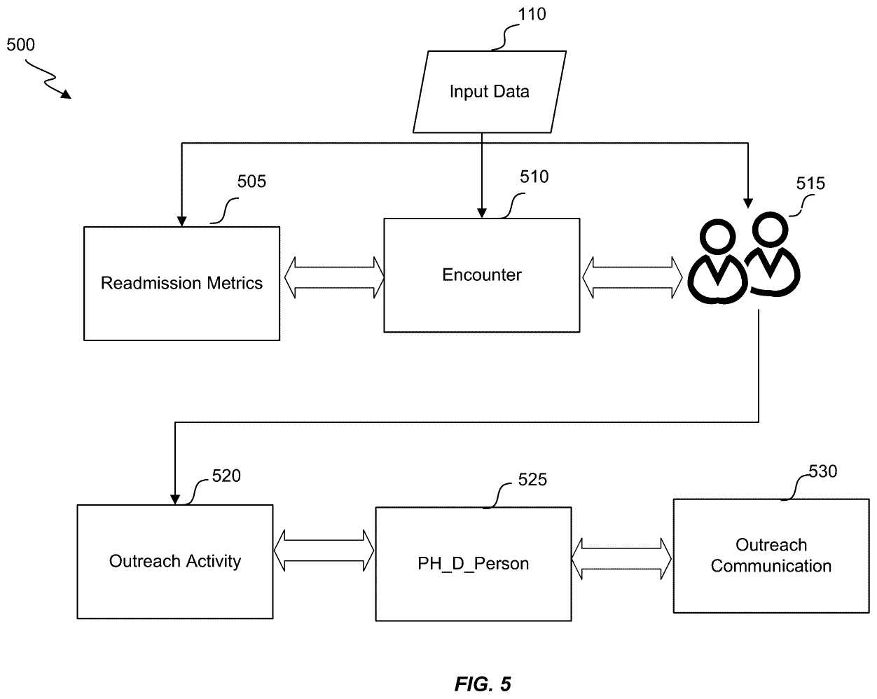

FIG. 5 illustrates a process flow diagram of a process 500 for tracking outreach communications and subsequent visits, according to an exemplary embodiment. The input data 110 may include data stored and/or managed by a medical facility network, medical facility, department, etc. The process 500 can track, for each encounter: an identity of the subject 515, whether it was a readmission related to a prior visit based on the readmission metrics 505 and information about the encounter 510 (e.g., what test(s) were performed, test results, a medical provider's assessment, any prescription provided, etc.)

Similarly, data associated with outreach activity 520 can indicate when, how, and to whom outreach communications were provided. For example, data associated with the outreach activity 520 can indicate that an alert in a medical facility-associated software application was sent to a given subject on a given date, recommending that they visit a particular cardiac department within three days.

The process can detect that the person to whom the outreach communication 530 was sent is the same person who had the encounter (e.g., after the outreach communication). Thus, it can be inferred that the outreach was successful in prompting the person to seek medical care. It can be evaluated whether the encounter occurred at the recommended particular cardiac department. The logic for mapping an encounter or visit to outreach calls is essentially achieved using the stages described below.

Identify the call records using ‘serial_no’ and date & time of visit. These two variables are given a new identifier specific to each serial number referred to as “Communication_id”. The subject visits can be distributed across multiple departments as many subjects could have comorbidities like a cardiac subject could also be suffering from type 1 diabetes. Hence develop two features, firstly an identifier based on department visit and subject id resulting in a new feature called “department_subject_id”.

Secondly, to identify the interdependence of departments for final diagnosis, for this we generate a variable by counting the number of visits for each subject. A data scrapping is performed across all departments to count the visits per subject across unique encounter, this new calculated feature is referred to as “department_encounter_subject_id.

Calculate a feature called “department_start_call” using the subject identifier by utilizing the above 3 features. This feature enables healthcare professionals to identify each unique communication for a department and enables in identifying subjects whose visitation are dependent on other departments.

Calculate whether the outreach call for unique the subject was successful using date filtering and ‘communication_id’ for each client location.

This information can then be used during a self-learning process to further train the machine-learning model as to what types of outreach efforts are likely to lead to target allocations of subject loads across departments, medical facilities, and/or time periods.

### Exemplary Advantages

The efficient and automated approaches disclosed herein result in many advantages, such as:
Medical facility outcomes: Medical facilities will have a fair idea about the number of subject visits and strategies which are unique to each department and these strategies help medical facilities to manage their available resources more dynamically hence it provides a competitive advantage over other medical facilities, and it eventually increases their profit and operational efficiency.
Subject outcomes: Subjects will receive better healthcare services that are delivered on time with better efficiency and the quality of the healthcare infrastructure available. This improves the subject's health.
Organization outcomes: This innovation reduces the overall stress on resources, enabling healthcare administrators to match demand across various departments and proactively take steps to schedule healthcare professionals. Thus, resources can be dynamically and efficiently adjusted. Such adjustment can further be based on automated interconnectivity between different locations of hospitals and clinics, to provide resource adjustments in a manner not feasible before.
Financial planning: This process enables healthcare professionals to provide holistic budgets specific to demand and supply metrics. Hence administrators can provide more accurate budget estimates and more efficiently allocate proper resources.
Scheduling: Based on the forecasts, sudden demand surges can be predicted and managed by allocating healthcare professionals accurately for each location or department.

Further, various departments also may realize department-specific advantages. For example:
Surgery department: the surgery department can plan well in advance the availability of medical staff, surgeons, experts like anesthesiologists and medicines for performing surgery.
Outpatient department: several outpatient services like consultation, investigation, procedures, and specialty services can be planned well in advance with the available resources.
Inpatient department: several inpatient services like wards and rooms, nurses' station and dietary services can be planned well in advance with the available resources.
Emergency department: the emergency department will have a fair idea about the number of inflows subjects which can be used to arrange necessary medical equipment and medical staff.
Radiology department: the doctors can plan well in advance the availability of scanning equipment and number of scans to be performed.
Pharmacy department: as hospitals will have a fair idea about the number of subject visits, they can do dynamic management of medicines.
Pathology department: the various laboratories within the pathology department can plan well in advance the availability of resources.
Blood bank: as major hospitals will have a blood bank unit; the bank does better management of availability of blood with them.

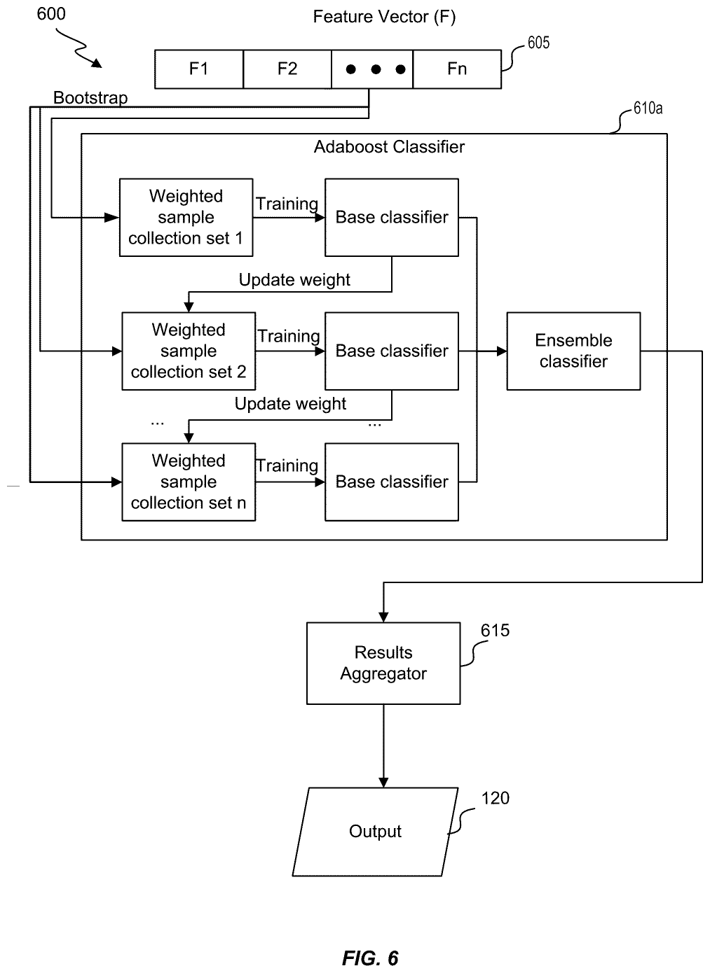

FIG. 6 illustrates a process flow diagram of a process for using feature vector to train the machine-learning model by way of system 600. The system 600 includes a Feature vector (F) 605, an Adaptive Boosting (Adaboost) classifier 610a, a result aggregator 615 and an output 120. The extracted features and generated derived features are collated into a candidate feature vector which may be used to train a machine-learning model and/or underlying algorithms. The example embodiment uses an Adaboost classifier 610a to generate prediction of number of subjects visiting a medical facility within a certain period of time using Adaptive Boosting (Adaboost) technique. Adaptive Boosting technique is an ensemble machine learning algorithm. It is a supervised learning algorithm that is used to classify data by combining multiple weak or base learners (e.g., decision trees) into a strong learner. The Adaboost model works by weighing the instances in the training dataset based on the accuracy of previous classifications. The model is first trained using the training dataset composed of candidate feature vectors and then builds a second model using the training dataset to correct the faults in the first model. The process is repeated until the errors of the model are reduced and the input dataset is accurately predicted. The model may use decision trees with one level of decision tree nodes (decision trees with just one split) as estimator. The technique constructs a model and assigns equal weights to all data points in the training input dataset. The model then applies larger weights to incorrectly categorized points than the weights assigned to correctly classified points of the dataset. In the next iteration, all points with greater weights are given more weight for training the model. It is continued to train models until a smaller error is returned. The model's ability to weight instances based on previous classification makes it robust to noisy and imbalanced dataset, and it is computationally efficient and less prone to overfitting.

The model may predict for every subject whether they will visit a particular medical facility within a specified period of time. Result aggregator result 615 receives individual predictions of each subject and aggregate the number of subjects predicted to visit a particular department in a particular medical facility. The output 120 is generated which can be communicated to the administration staff of the medical facility. When the medical facility gets an estimate about the number of subjects visits, they can plan for the availability of medicines, resources, essential staff, and other medical equipment's to deliver proper care management.

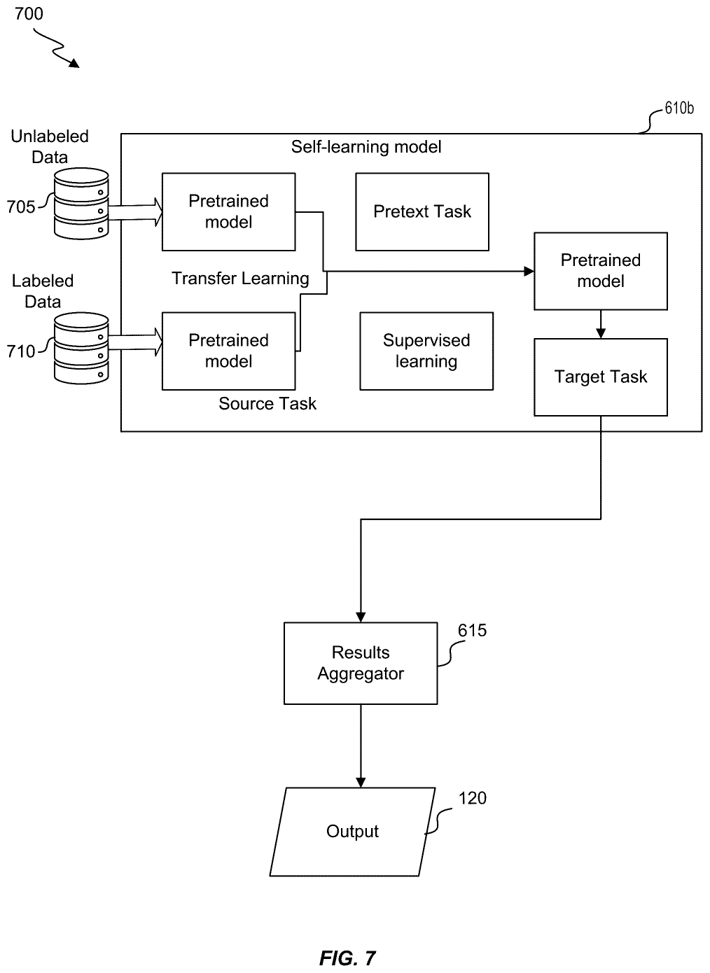

FIG. 7 illustrates another example process flow of the present disclosure, where a self-learning machine learning model is used to generate the prediction. The example embodiment may be implemented by system 700 that includes unlabeled data 705, labelled data 710, a Self-learning model 610b, a result aggregator 615 and the output 120. The labelled data 710 comprises of the dataset with target labels, where target label can include the indication of a subject visiting a particular medical facility within a specified period of time after an outreach communication happened. The labelled data can be used to train supervised machine learning models to predict the number of subjects visiting the particular medical facility within a specified period of time. The unlabeled data does not contain labels identifying characteristics, properties, or classification. Unlabeled data has no labels or targets to predict, only features to represent them. Self-learning model 610b can train itself using labeled and unlabeled data. It analyzes the dataset for patterns that it can draw conclusions from. The machine-learning model once deployed can be optimized by training them on data that becomes more available over time. As new data becomes available every day the self-learning model can improve on an ongoing basis by systematically recording the design characteristics, test conditions, and test results. Self-learning model enables the system and method to be sufficiently flexible and adaptable to detect and incorporate trends or differences in the underlying subject data that may affect the predictive accuracy of a given algorithm. The model may periodically retrain a selected predictive model for an improved accurate outcome. Self-learning models may employ various methods such as pseudo-labeling and two classifiers self-training. Pseudo-labeling generates labels for unlabeled data and use the new unlabeled data to retrain the model with more information. For the two-classifiers, at each step, a classifier is trained on the available data, and then predict the next batch of new data. Then the classifier is switched, and the process is repeated multiple times. The machine-learning model may be used to predict the likelihood of a number of successful outreaches and subject visits in a specified period of time. As the ML model keeps learning from the everyday data it also tends to improve its performance over time. When the medical facility gets an estimate about the number of subjects visits, they can plan for the availability of medicines, resources, essential staff, and other medical equipment's to deliver efficient care management. The model may generate prediction of every subject whether they will visit a particular medical facility within a specified period of time. Result aggregator 615 receives individual predictions of each subject and aggregate the number of subjects predicted to visit a particular department in a particular medical facility. The output 120 is generated which can be communicated to the administration staff of the medical facility.

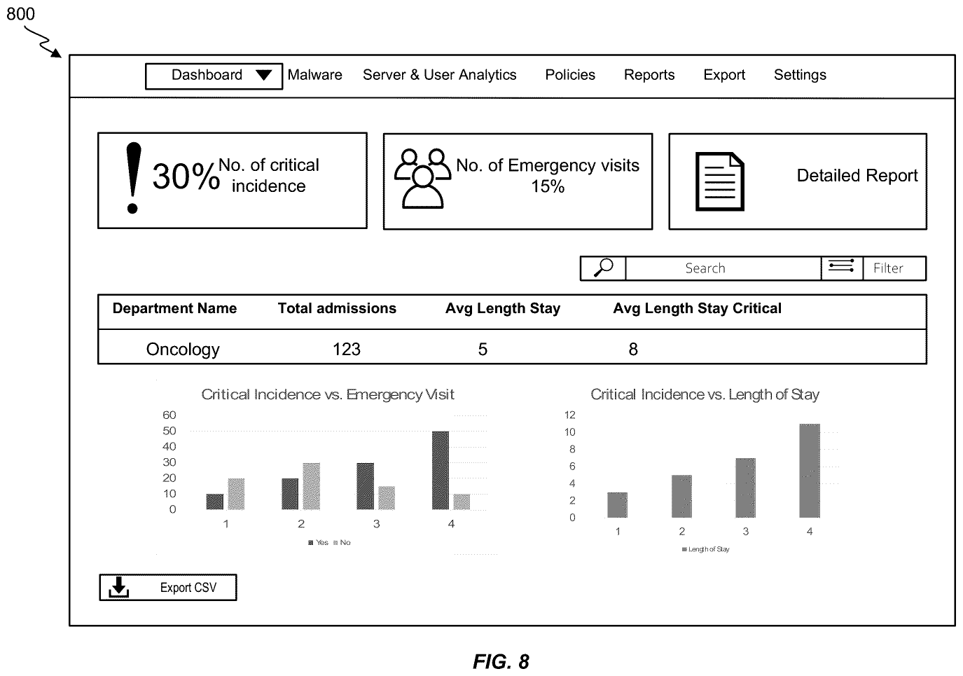

FIG. 8 illustrates an example embodiment of an application Graphical User Interface (GUI) 800 generated by system 100. The example application interface can be configured based on predicted readmissions that will occur within an emergency department of a particular medical facility within a three-day time span. The predictions are generated using by transforming, for each subject who received an outreach communication recommending that the subject seek medical care at the emergency department, a feature set for the subject using a trained machine-learning model. A dashboard 800 may display the detailed record of each subject after the analysis of the data has been effectively performed by using machine-learning models.

The dashboard may descriptively hold various sections where the subject's outreach statistics information may be displayed elaboratively. The number of critical incidence block displays the percentage of critical incidence for a particular department at the medical facility along with detailed report of subjects incurring the critical incidences and number of emergency visits. As the name indicates, the detailed report section shows the descriptive report of each subject of a set of subjects including the previous EMR and the updated health records along with the subject ID assigned to each subject. Subject ID displays the unique ID that each subject gets in the initial phase of analysis to maintain confidentiality and the integrity of each subject's data. The panel also shows a table showing the records of each department in the medical facility. The table may include a column like department name, total number of admissions in the respective department, average length of the stay of the subject and average length of stay in critical care unit. The dashboard may also display the comparison of critical incidence numbers vs. number of emergency visit for a particular medical facility in a graphical chart. The two graphs show a predicted number of subjects who will arrive at the emergency department within the three-day time span, will not be deemed to have a critical incident, but will be readmitted and comparison of the critical incidences vs. predicted length of stay for the subjects. Further, the data is separated to predict how many of those subjects will have stays of various numbers of days. When the medical facility gets an estimate about the number of subjects visits, they can plan for the availability of medicines, resources, essential staff, and other medical equipment to deliver proper care management.

The advantage of this dashboard is that it displays the detailed insight of the medical facility predicted usage, which clearly informs the facility administration stuff about the predicted load of the different departments and the resources can be arranged accordingly. The interface can allow a medical facility to consider whether it has sufficient resources to account for such emergency visits and subject stays.

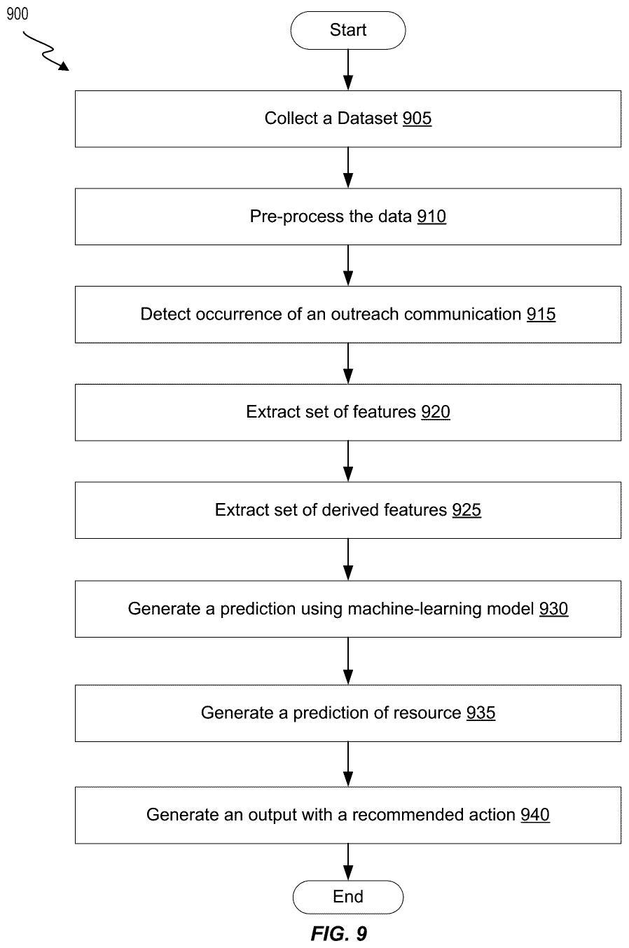

FIG. 9 illustrates an example flow of a method for obtaining the output result of resource usage prediction using a machine-learning model trained on a set of features in accordance with some embodiments of the present disclosure. Referring to FIG. 9, at block 905, a dataset is collected from a variety of data sources. The data sources include data from Electronic Medical Records (EMR), Electronic Health Records (EHR), or may also include medical registries. Dataset may include text data or time series data. At block 910, the collected dataset is preprocessed to enhance the quality of the data and ensure data consistency. Data preprocessing includes a range of operations, including noise reduction, data normalization, and data cleaning. At block 915, an occurrence of an outreach communication is detected from the input dataset. To track outreach communication and risk management of the subject, complete and current health records of the subjects being served are required. Outreach involves a systematic process for checking on referrals, lab tests, and prescribed medications. The health centers also keeps information for the subjects who seek follow-up services at a particular medical facility and routinely maintains the occurrence of an outreach communication. At block, 920 feature extraction is performed to extract a relevant set of features from the dataset. These features are qualities or traits found in the data that can be used to predict the number of successful outreaches. By concentrating on the relevant features, feature extraction process extracts the features from the dataset that may improves the analytical readability of the machine-learning model by improving the performance of the model. At block 925, derived features are generated from one or more extracted set of features. For instance, the variable “incident_critical” is derived using the “type of pre-conditions” and “emergency visit” of the subject. On these two variables, logical rules are applied to determine the subject's risk status. Derived features can enhance the predictive capabilities of the model by capturing complex relationships within the dataset. At block 930, ML model 230 is used to predict the number of subjects visiting the medical facility within a specific period of time. The model analyzes the preprocessed data, the extracted set of features and derived set of features to generate the prediction. The predicted likelihood of successful outreaches can guide the decision-making process in an informed manner. Machine-learning models can include Random Forests, Support Vector Machine, AdaBoost, Ensemble models or self-learning models etc. At block 935, the ML model may also predict the resource usage of a particular medical facility. The prediction may provide the information that identifies a current availability status of the resources such as healthcare workers, that identifies or indicates durations of time until the respective workers will become available to perform a healthcare task (e.g. available now, available in 30 minutes, available between hours 1000 and 1100), amount of time the respective workers have available to perform certain tasks (e.g. based on scheduling constraints, shift end time, etc.). The prediction of resource usage/availability can include information that identifies known or expected timeslots over a defined upcoming timeframe (e.g. the next 24 hours, the next week, the next month, etc.) in which one or more healthcare workers are available to perform healthcare tasks in specific medical facilities. In traditional approaches, the medical facilities do not have any idea about the number of subject visits, and it assumes the number of subjects visits based on previous data which results in inaccurate estimates. Hence the medical facilities tend to overutilize or underutilize the available resources (medicines, staff etc.) which results in delivering improper healthcare services to the subjects. Using the prediction of the ML model, a strategy can be devised which is unique to each department and using this strategy each department can operate to their full potential and provide quality healthcare services. It may help in proactive management of the number of outreach calls, enabling better capacity prediction for future trends and optimal allocation and utilization of resources for the medical facilities. At block 940, an output with a recommended action is generated. Recommended action may include devising department specific strategy. The strategy may (for example) indicate whether, when and/or how to change a resource allocation for a given department in view of the predicted number. For example, if the predicted number is relatively high, a computer interface may transmit or display a recommendation that a new supply or medication resources may be purchased, and/or more human resources may be recruited (e.g., by reassigning a medical professional from one department to another at least temporarily, by recruiting a contract medical professional, by requesting that a medical professional in the department work overtime, etc.). Whether, when and/or how the resource allocation is changed in response to a given predicted number may vary across departments (e.g., given that a skill level or experience required may vary across departments).

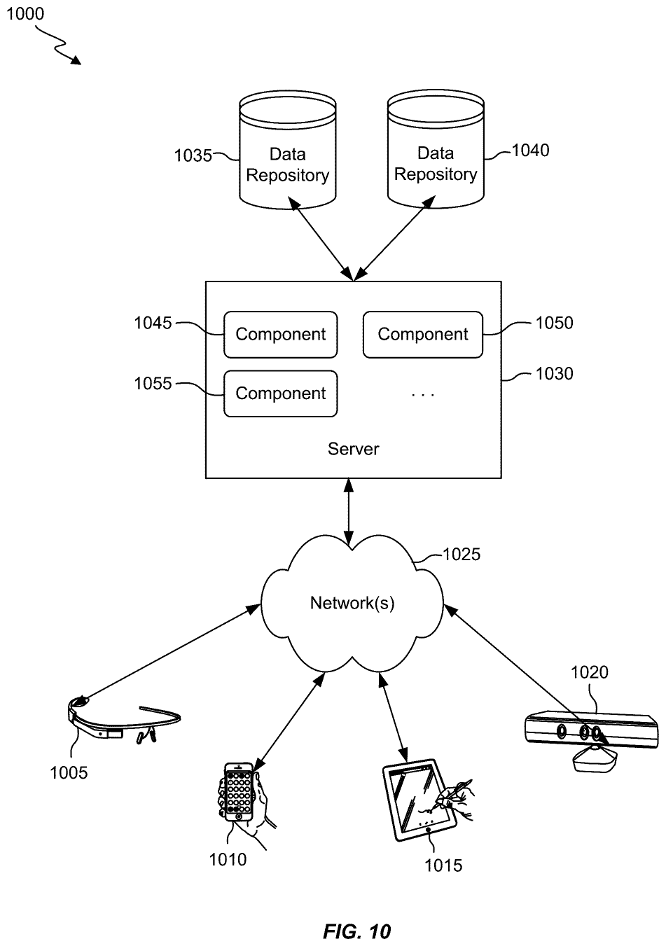

FIG. 10 depicts a simplified diagram of a distributed system 1000 for computer-implemented method 100 of FIG. 1. In the illustrated embodiment, distributed system 1000 includes one or more subject computing devices 1005, 1010, 1015, and 1020, coupled to a server 1030 via one or more network(s) 1025. Subjects computing devices 1005, 1010, 1015, and 1020 may be configured to execute one or more applications.

In various aspects, server 1030 may be adapted to run one or services or software applications that enable techniques for predicting resource usage from a dataset gathered from different data sources. In certain aspects, server 1030 may also provide other services or software applications that can include non-virtual and virtual environments. In some respects, these services may be offered as web-based or cloud services, such as under a Software as a Service (SaaS) model to the users of subject computing devices 1005, 1010, 1015, and/or 1020. Users operating subject computing devices 1005, 1010, 1015, and/or 1020 may, in turn, utilize one or more subject applications to interact with server 1030 to utilize the services provided by these components. Furthermore, subject computing devices 1005, 1010, 1015, and/or 1020 may, in turn, utilize one or more subject applications for predicting resource usage.

In the configuration depicted in FIG. 10, server 1030 may include one or more components 1045, 1050, and 1055 that implement the functions performed by server 1030. These components may include software components that may be executed by one or more processors, hardware components, or combinations thereof. It may be appreciated that various system configurations are possible, which may be different from distributed system 1000. The embodiment shown in FIG. 10 is thus one example of a distributed system for implementing an embodiment system and is not intended to be limiting.

Users may use subject computing devices 1005, 1010, 1015, and/or 1020 for predicting resource usage from the dataset collected from a variety of data sources using various machine-learning models such as Random Forest model, Support Vector Machine model, AdaBoost model, etc. in accordance with the teachings of this disclosure. A subject device may provide an interface that enables a user of the subject device to interact with the subject device. The subject device may also output information to the user via this interface. Although FIG. 10 depicts only four subject computing devices, any number of subject computing devices may be supported.

The subject devices may include various types of computing systems such as portable handheld devices, general purpose computers such as personal computers and laptops, workstation computers, wearable devices, gaming systems, thin subjects, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone®), tablets (e.g., iPad®), personal digital assistants (PDAs), and the like. Wearable devices may include Google Glass® head-mounted displays and other devices. Gaming systems may include various handheld gaming devices, Internet-enabled gaming devices (e.g., a Microsoft Xbox® gaming console with or without a Kinect® gesture input device, Sony PlayStation® system, various gaming systems provided by Nintendo®, and others), and the like. The subject devices may be capable of executing various applications such as various Internet-related apps and communication applications (e.g., E-mail applications, short message service (SMS) applications) and may use various communication protocols.

Network(s) 1025 may be any type of network familiar to those skilled in the art that can support data communications using any of a variety of available protocols, including without limitation TCP/IP (transmission control protocol/Internet protocol), SNA (systems network architecture), IPX (Internet packet exchange), AppleTalk®, and the like. Merely by way of example, network(s) 1025 can be a Local Area Network (LAN), network based on Ethernet, Token-Ring, a Wide-Area Network (WAN), the Internet, a virtual network, a Virtual Private Network (VPN), an intranet, an extranet, a Public Switched Telephone Network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Server 1030 may include one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 1030 can include one or more virtual machines running virtual operating systems, or other computing architectures involving virtualization such as one or more flexible pools of logical storage devices that can be virtualized to maintain virtual storage devices for the server. In various aspects, server 1030 may be adapted to run one or more services or software applications that provide the functionality described in the foregoing disclosure.

The computing systems in server 1030 may run one or more operating systems including any of those discussed above, as well as any commercially available server operating system. Server 1030 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation, those commercially available from Oracle®, Microsoft®, Sybase®, IBM® (International Business Machines), and the like.

In some implementations, server 1030 may include one or more applications to implement various machine-learning algorithms. The data in 110 of FIG. 1 may include data of various forms such as text data, audio data, time-series data, and real-time data. As an example, in a case where the data samples are text or image that may include but are not limited to, Twitter® feeds, Facebook® updates, or real-time updates received from one or more third-party information sources and continuous data streams, which may include real-time events related to sensor data applications, financial tickers, network performance measuring tools (e.g., network monitoring and traffic management applications), clickstream analysis tools, automobile traffic monitoring, and the like. Server 1030 may also include one or more applications to display the output of various processes of computer-implemented method 100 via one or more display devices of subject computing devices 1005, 1010, 1015, and 1020.

Distributed system 1000 may also include one or more data repositories 1035, and 1040. These data repositories may be used to store data in 110 in database and other information in certain aspects. Data repositories 1035, and 1040 may reside in a variety of locations. For example, a data repository used by server 1030 may be local to server 1030 or may be remote from server 1030 and in communication with server 1030 via a network-based or dedicated connection. Data repositories 1035, and 1040 may be of different types. In certain aspects, a data repository used by server 1030 may be a database, for example, a relational database, such as databases provided by Oracle Corporation® and other vendors. One or more of these databases may be adapted to enable storage, update, and retrieval of data to and from the database in response to Structured Query Language (SQL)-formatted commands.

In certain aspects, one or more data repositories 1035, and 1040 may also be used by applications to store application data. The data repositories used by applications may be of different types such as, for example, a key-value store repository, an object store repository, or a general storage repository supported by a file system.

In certain aspects, the techniques for predicting resource usage from the dataset collected from a variety of data sources using various machine-learning models described in this disclosure may be offered as services via a cloud environment. FIG. 10 is a simplified block diagram of a cloud-based system environment in which various services of server 1030 of FIG. 10 may be offered as cloud services, in accordance with certain aspects. In the embodiment depicted in FIG. 10, subject computing devices 1005 may provide one or more cloud services that may be requested by users using one or more subject computing devices 1010, 1015, and 1020. Subject computing devices 1005 may comprise one or more computers and/or servers that may include those described for server 1030. The computers in subject computing devices 1005 may be organized as general-purpose computers, specialized server computers, server farms, server clusters, or any other appropriate arrangement and/or combination.

Network(s) 1025 may facilitate communication and exchange of data between subject computing devices 1010, 1015, and 1020 and subject computing devices 1005. Network(s) 1025 may include one or more networks. The networks may be of the same or different types. Network(s) 1025 may support one or more communication protocols, including wired and/or wireless protocols, for facilitating communications.

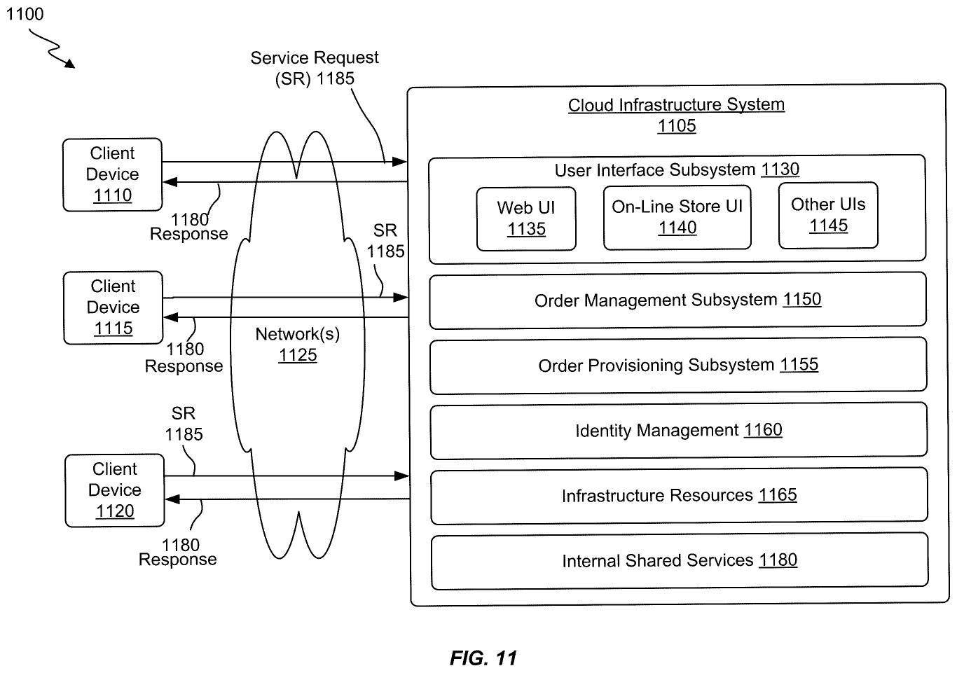

The embodiment depicted in FIG. 11 is only one example of a cloud infrastructure system and is not intended to be limiting. It should be appreciated that, in some other respects, cloud infrastructure system 1105 may have more or fewer components than those depicted in FIG. 11, may combine two or more components, or may have a different configuration or arrangement of components. For example, although FIG. 11 depicts three subject computing devices, any number of subject computing devices may be supported in alternative aspects.

The term cloud service is generally used to refer to a service that is made available to users on demand and via a communication network such as the Internet by systems (e.g., cloud infrastructure system 1105) of a service provider. Typically, in a public cloud environment, servers and systems that make up the cloud service provider's system are different from the subject's own on-premises servers and systems. The cloud service provider's systems are managed by the cloud service provider. Subjects can thus avail themselves of cloud services provided by a cloud service provider without having to purchase separate licenses, support, or hardware and software resources for the services. For example, a cloud service provider's system may host an application, and a user may, via a network 1125 (e.g., the Internet), on demand, order and use the application without the user having to buy infrastructure resources for executing the application. Cloud services are designed to provide easy, scalable access to applications, resources, and services. Several providers offer cloud services. For example, several cloud services are offered by Oracle Corporation® of Redwood Shores, California, such as middleware services, database services, Java cloud services, and others.

In certain aspects, cloud infrastructure system 1105 may provide one or more cloud services using different models such as under a Software as a Service (SaaS) model, a Platform as a Service (PaaS) model, an Infrastructure as a Service (IaaS) model, and others, including hybrid service models. Cloud infrastructure system 1105 may include a suite of applications, middleware, databases, and other resources that enable the provision of the various cloud services.

A SaaS model enables an application or software to be delivered to a subject over a communication network like the Internet, as a service, without the subject having to buy the hardware or software for the underlying application. For example, a SaaS model may be used to provide subjects access to on-demand applications that are hosted by cloud infrastructure system 1105. Examples of SaaS services provided by Oracle Corporation® include, without limitation, various services for human resources/capital management, subject relationship management (CRM), enterprise resource planning (ERP), supply chain management (SCM), enterprise performance management (EPM), analytics services, social applications, and others.

An IaaS model is generally used to provide infrastructure resources (e.g., servers, storage, hardware, and networking resources) to a subject as a cloud service to provide elastic compute and storage capabilities. Various IaaS services are provided by Oracle Corporation®.

A PaaS model is generally used to provide, as a service, platform, and environment resources that enable subjects to develop, run, and manage applications and services without the subject having to procure, build, or maintain such resources. Examples of PaaS services provided by Oracle Corporation® include, without limitation, Oracle Java Cloud Service (JCS), Oracle Database Cloud Service (DBCS), data management cloud service, various application development solutions services, and others.

Cloud services are generally provided on an on-demand self-service basis, subscription-based, elastically scalable, reliable, highly available, and secure manner. For example, a subject, via a subscription order, may order one or more services provided by cloud infrastructure system 1105. Cloud infrastructure system 1105 then performs processing to provide the services requested in the subject's subscription order. Cloud infrastructure system 1105 may be configured to provide one or even multiple cloud services.

Cloud infrastructure system 1105 may provide cloud services via different deployment models. In a public cloud model, cloud infrastructure system 1105 may be owned by a third-party cloud services provider and the cloud services are offered to any general public subject, where the subject can be an individual or an enterprise. In certain other aspects, under a private cloud model, cloud infrastructure system 1105 may be operated within an organization (e.g., within an enterprise organization) and services provided to subjects that are within the organization. For example, the subjects may be various departments of an enterprise, such as the Human Resources department, the payroll department, etc. or even individuals within the enterprise. In certain other aspects, under a community cloud model, the cloud infrastructure system 1105 and the services provided may be shared by several organizations in a related community. Various other models, such as hybrids of the above-mentioned models may also be used.

Subject computing devices 1110, 1115, and 1120 may be of several types (such as cloud infrastructure system 1105, 1110, 1115, and 1120 depicted in FIG. 11) and may be capable of operating one or more subject applications. A user may use a subject device to interact with Cloud Infrastructure System 1105, such as to request a service provided by Cloud Infrastructure System 1105.

As depicted in the embodiment in FIG. 11, cloud infrastructure system 1105 may include infrastructure resources 1175 that can be utilized for facilitating the provision of various cloud services offered by cloud infrastructure system 1105. These services include 910, 915, 920, 925, 930, 935, and 940 as shown in FIG. 9. Infrastructure resources 1175 may include, for example, processing resources, storage or memory resources, networking resources, and the like.

In certain aspects, to facilitate efficient provisioning of these resources for supporting the various cloud services provided by cloud infrastructure system 1105 for different subjects, the resources may be bundled into sets of resources or resource modules (also referred to as “pods”). Each resource module or pod may comprise a pre-integrated and optimized combination of resources of one or more types. In certain aspects, different pods may be pre-provisioned for different types of cloud services. For example, a first set of pods may be provisioned for a database service, a second set of pods, which may include a different combination of resources than a pod in the first set of pods, may be provisioned for Java service, and the like. For some services, the resources allocated for provisioning the services may be shared between the services.

Cloud infrastructure system 1105 may itself internally use services 1170 that are shared by different components of cloud infrastructure system 1105 and which facilitate the provisioning of services by cloud infrastructure system 1105. These internal shared services may include, without limitation, a security and identity service, an integration service, an enterprise repository service, an enterprise manager service, a virus scanning and whitelist service, a high availability, backup and recovery service, service for enabling cloud support, an email service, a notification service, a file transfer service, and the like.

Cloud infrastructure system 1105 may comprise multiple subsystems. These subsystems may be implemented in software, or hardware, or combinations thereof. As depicted in FIG. 11, the subsystems may include a user interface subsystem 1130 that enables users or subjects of cloud infrastructure system 1105 to interact with cloud infrastructure system 1105. User interface subsystem 1130 may include various interfaces such as a web interface 1135, an online store interface 1140 where cloud services provided by cloud infrastructure system 1105 are advertised and are purchasable by a consumer, and other interfaces 1145. For example, a subject may, using a subject device, request (service request 1175) one or more services provided by cloud infrastructure system 1105 using one or more of interfaces 1135, 1140, and 1145. For example, a subject may access the online store, browse cloud services offered by cloud infrastructure system 1105, and place a subscription order for one or more services offered by cloud infrastructure system 1105 that the subject wishes to subscribe to. The service request may include information identifying the subject and one or more services that the subject desires to subscribe to. For example, a subject may place a subscription order for a Chabot related service offered by cloud infrastructure system 1105. As part of the order, the subject may provide information identifying for input (e.g., utterances).

In certain aspects, such as the embodiment depicted in FIG. 11, cloud infrastructure system 1105 may comprise an Order Management Subsystem (OMS) 1150 that is configured to process the new order. As part of this processing, OMS 1150 may be configured to: create an account for the subject, if not done already; receive billing and/or accounting information from the subject that is to be used for billing the subject for providing the requested service to the subject; verify the subject information; upon verification, book the order for the subject; and orchestrate various workflows to prepare the order for provisioning.

Once properly validated, OMS 1150 may then invoke Order Provisioning Subsystem (OPS) 1155 that is configured to provision resources for the order including processing, memory, and networking resources. The provisioning may include allocating resources for the order and configuring the resources to facilitate the service requested by the subject order. The manner in which resources are provisioned for an order and the type of the provisioned resources may depend upon the type of cloud service that has been ordered by the subject. For example, according to one workflow, OPS 1155 may be configured to determine the particular cloud service being requested and identify a number of pods that may have been pre-configured for that particular cloud service. The number of pods that are allocated for an order may depend upon the size/amount/level/scope of the requested service. For example, the number of pods to be allocated may be determined based upon the number of users to be supported by the service, the duration of time for which the service is being requested, and the like. The allocated pods may then be customized for the particular requesting subject for providing the requested service.

Cloud infrastructure system 1105 may send a response or notification 1190 to the requesting subject to indicate when the requested service is now ready for use. In some instances, information (e.g., a link) may be sent to the subject that enables the subject to start using and availing the benefits of the requested services.

Cloud infrastructure system 1105 may provide services to multiple subjects. For each subject, cloud infrastructure system 1105 is responsible for managing information related to one or more subscription orders received from the subject, maintaining subject data related to the orders, and providing the requested services to the subject. Cloud infrastructure system 1105 may also collect usage statistics regarding a subject's use of subscribed services. For example, statistics may be collected for the amount of storage used, the amount of data transferred, the number of users, and the amount of system up time and system down time, and the like. This usage information may be used to bill the subject. Billing may be done, for example, on a monthly cycle.

Cloud infrastructure system 1105 may provide services to multiple subjects in parallel. Cloud infrastructure system 1105 may store information for these subjects, including possibly proprietary information. In certain aspects, cloud infrastructure system 1105 includes an identity management subsystem Identity Management Subsystem (IMS) 1170 that is configured to manage the subject's information and provide the separation of the managed information such that information related to one subject is not accessible by another subject. IMS 1170 may be configured to provide various security-related services such as identity services, such as information access management, authentication and authorization services, services for managing subject identities and roles and related capabilities, and the like.

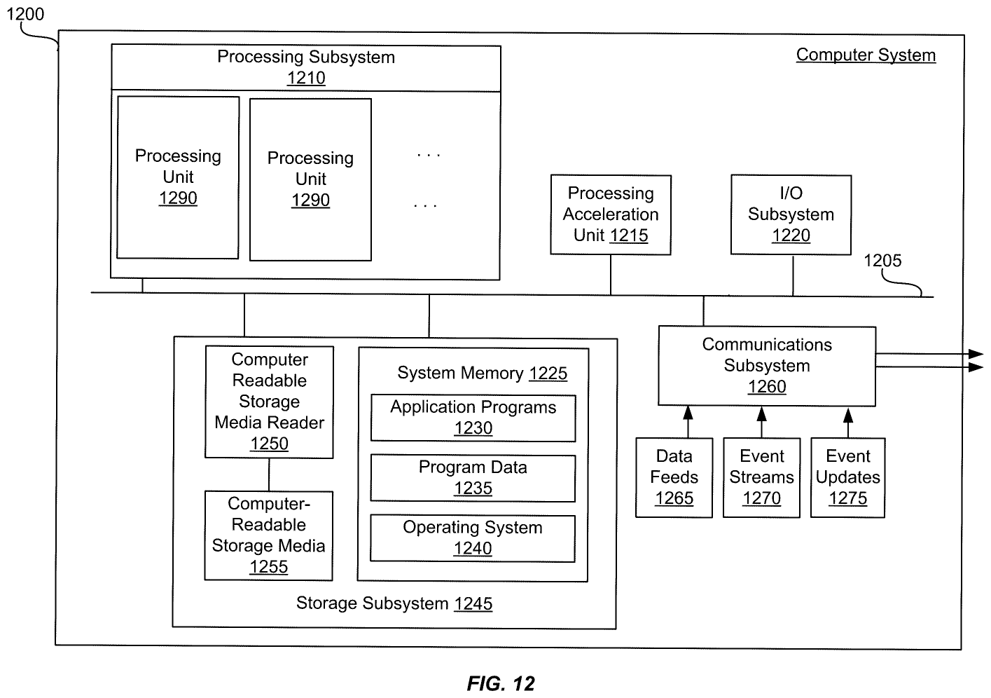

FIG. 12 illustrates an exemplary computer system 1200 that may be used to implement certain aspects of computer-implemented method 100 for predicting resource usage. For example, in some respects, computer system 1200 may be used to implement any of the systems for predicting resource usage from the dataset collected from a variety of data sources using various machine-learning models shown in FIG. 1 and various servers and computer systems described above. As shown in FIG. 12, computer system 1200 includes various subsystems including a processing subsystem 1210 that communicates with a few other subsystems via a bus subsystem 1205. These other subsystems may include a processing acceleration unit 1215, and I/O subsystem 1220, a storage subsystem 1245, and a communications subsystem 1270. Storage subsystem 1245 may include non-transitory computer-readable storage media including storage media 1255 and a system memory 1225.

Bus subsystem 1205 provides a mechanism for letting the various components and subsystems of computer system 1200 communicate with each other as intended. Although bus subsystem 1205 is shown schematically as a single bus, alternative aspects of the bus subsystem may utilize multiple buses. Bus subsystem 1205 may be any of several types of bus structures including a memory bus or memory controller, a peripheral bus, a local bus using any of a variety of bus architectures, and the like. For example, such architectures may include an Industry Standard Architecture (ISA) bus, a Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P13127.1 standard, and the like.

Processing subsystem 1210 controls the operation of computer system 1200 and may comprise one or more processors, Application Specific Integrated Circuits (ASICs), or Field Programmable Gate Arrays (FPGAs). The processors may include single-core, or multicore processors. The processing resources of computer system 1200 can be organized into one or more processing units 1290, 1280, etc. A processing unit may include one or more processors, one or more cores from the same or different processors, a combination of cores and processors, or other combinations of cores and processors. In some embodiments, processing subsystem 1210 can include one or more special-purpose co-processors such as graphics processors, digital signal processors (DSPs), or the like. In some embodiments, some or all of the processing units of processing subsystem 1210 can be implemented using customized circuits, such as ASICs, or FPGAs.

In some embodiments, the processing units in processing subsystem 1210 can execute instructions stored in system memory 1225 or on computer-readable storage media 1255. In various aspects, the processing units can execute a variety of programs or code instructions and can maintain multiple concurrently executing programs or processes. At any given time, some, or all of the program code to be executed can be resident in system memory 1225 and/or on computer-readable storage media 1255 including potentially on one or more storage devices. Through suitable programming, processing subsystem 1210 can provide various functionalities described above. In instances where computer system 1200 is executing one or more virtual machines, one or more processing units may be allocated to each virtual machine.

In certain aspects, a processing acceleration unit 1215 may optionally be provided for performing customized processing or for off-loading some of the processing performed by processing subsystem 1210 to accelerate the overall processing performed by computer system 1200.

I/O subsystem 1220 may include devices and mechanisms for inputting information to computer system 1200 and/or for outputting information from or via computer system 1200. In general, use of the term input device is intended to include all possible types of devices and mechanisms for inputting information to computer system 1200. User interface input devices may include, for example, a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may also include motion sensing and/or gesture recognition devices such as the Microsoft Kinect® motion sensor that enables users to control and interact with an input device, the Microsoft Xbox® 370 game controller, devices that provide an interface for receiving input using gestures and spoken commands. User interface input devices may also include eye gesture recognition devices such as the Google Glass® blink detector that detects eye activity (e.g., “blinking” while taking pictures and/or making a menu selection) from users and transforms the eye gestures as inputs to an input device (e.g., Google Glass®). Additionally, user interface input devices may include voice recognition sensing devices that enable users to interact with voice recognition systems (e.g., Siri® navigator) through voice commands.

Other examples of user interface input devices include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, and medical ultrasonography devices. User interface input devices may also include, for example, audio input devices such as MIDI keyboards, digital musical instruments, and the like.

In general, use of the term output device is intended to include all possible types of devices and mechanisms for outputting information from computer system 1200 to a user or other computer. User interface output devices may include a display subsystem, indicator lights, or non-visual displays such as audio output devices, etc. The display subsystem may be a Cathode Ray Tube (CRT), a flat-panel device, such as that using a Liquid Crystal Display (LCD) or plasma display, a projection device, a touch screen, and the like. For example, user interface output devices may include, without limitation, a variety of display devices that visually convey text, graphics, and audio/video information such as monitors, printers, speakers, headphones, automotive navigation systems, plotters, voice output devices, and modems.

Storage subsystem 1245 provides a repository or data store for storing information and data that is used by computer system 1200. Storage subsystem 1245 provides a tangible non-transitory computer-readable storage medium for storing the basic programming and data constructs that provide the functionality of some aspects. Storage subsystem 1245 may store software (e.g., programs, code modules, instructions) that, when executed by processing subsystem 1210 provides the functionality described above. The software may be executed by one or more processing units of processing subsystem 1210. Storage subsystem 1245 may also provide a repository for storing data used in accordance with the teachings of this disclosure.

Storage subsystem 1245 may include one or more non-transitory memory devices, including volatile and non-volatile memory devices. As shown in FIG. 12, storage subsystem 1245 includes a system memory 1225 and a computer-readable storage media 1255. System memory 1225 may include a number of memories including a volatile main random-access memory (RAM) for storage of instructions and data during program execution and a non-volatile Read Only Memory (ROM) or flash memory in which fixed instructions are stored. In some implementations, a basic input/output system (BIOS), containing the basic routines that help to transfer information between elements within computer system 1200, such as during start-up, may typically be stored in the ROM. The RAM typically contains data and/or program modules that are presently being operated and executed by processing subsystem 1210. In some implementations, system memory 1225 may include multiple different types of memory, such as Static Random Access Memory (SRAM), Dynamic Random Access Memory (DRAM), and the like.

By way of example, and not limitation, as depicted in FIG. 12, system memory 1225 may load application programs 1230 that are being executed, which may include various applications such as Web browsers, mid-tier applications, Relational Database Management Systems (RDBMS), etc., program data 1235, and an operating system 1240. By way of example, operating system 1240 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, Palm® OS operating systems, and others.

Computer-readable storage media 1255 may store programming and data constructs that provide the functionality of some aspects. Computer-readable media 1255 may provide storage of computer-readable instructions, data structures, program modules, and other data for computer system 1200. Software (programs, code modules, instructions) that, when executed by processing subsystem 1210 provides the functionality described above, may be stored in storage subsystem 1245. By way of example, computer-readable storage media 1255 may include non-volatile memory such as a hard disk drive, a magnetic disk drive, an optical disk drive such as a CD ROM, Digital Video Disc (DVD), a Blu-Ray® disk, or other optical media. Computer-readable storage media 1255 may include, but is not limited to, Zip® drives, flash memory cards, Universal Serial Bus (USB) flash drives, secure digital (SD) cards, DVD disks, digital video tape, and the like. Computer-readable storage media 1255 may also include, Solid-State Drives (SSD) based on non-volatile memory such as flash-memory based SSDs, enterprise flash drives, solid state ROM, and the like, SSDs based on volatile memory such as solid state RAM, dynamic RAM, static RAM, Dynamic Random Access Memory (DRAM)-based SSDs, magneto resistive RAM (MRAM) SSDs, and hybrid SSDs that use a combination of DRAM and flash memory based SSDs.

In certain aspects, storage subsystem 1245 may also include a computer-readable storage media reader 1250 that can further be connected to computer-readable storage media 1255. Reader 1250 may receive and be configured to read data from a memory device such as a disk, a flash drive, etc.

In certain aspects, Computer System 1200 may support virtualization technologies, including but not limited to the virtualization of processing and memory resources. For example, computer system 1200 may provide support for executing one or more virtual machines. In certain aspects, Computer System 1200 may execute a program such as a hypervisor that facilitates the configuring and managing of the virtual machines. Each virtual machine may be allocated memory, compute (e.g., processors, cores), I/O, and networking resources. Each virtual machine generally runs independently of the other virtual machines. A virtual machine typically runs its own operating system, which may be the same as or different from the operating systems executed by other virtual machines executed by computer system 1200. Accordingly, multiple operating systems may potentially be run concurrently by Computer System 1200.

Communications subsystem 1270 provides an interface to other computer systems and networks. Communications subsystem 1270 serves as an interface for receiving data from and transmitting data to other systems from computer system 1200. For example, communications subsystem 1270 may enable computer system 1200 to establish a communication channel to one or more subject devices via the Internet for receiving and sending information from and to the subject devices. For example, the communication subsystem may be used to transmit a response to a user regarding the inquiry for a Chabot.

Communication subsystem 1270 may support both wired and/or wireless communication protocols. For example, in certain aspects, communications subsystem 1270 may include Radio Frequency (RF) transceiver components for accessing wireless voice and/or data networks (e.g., using cellular telephone technology, advanced data network technology, such as 3G, 4G or EDGE (enhanced data rates for global evolution), Wi-Fi (IEEE 1202.XX family standards, or other mobile communication technologies, or any combination thereof), Global Positioning System (GPS) receiver components, and/or other components. In some aspects communications subsystem 1270 can provide wired network connectivity (e.g., Ethernet) in addition to or instead of a wireless interface.

Communication subsystem 1270 can receive and transmit data in various forms. For example, in some embodiments, in addition to other forms, communications subsystem 1270 may receive input communications in the form of structured and/or unstructured data feeds 1275, event streams 1270, event updates 1275, and the like. For example, communications subsystem 1270 may be configured to receive (or send) data feeds 1275 in real-time from users of social media networks and/or other communication services such as Twitter® feeds, Facebook® updates, web feeds such as Rich Site Summary (RSS) feeds, and/or real-time updates from one or more third party information sources.

In certain aspects, communications subsystem 1270 may be configured to receive data in the form of continuous data streams, which may include event streams 1270 of real-time events and/or event updates 1275, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1270 may also be configured to communicate data from computer system 1200 to other computer systems or networks. The data may be communicated in various forms such as structured and/or unstructured data feeds 1275, event streams 1270, event updates 1275, and the like to one or more databases that may be in communication with one or more streaming data source computers coupled to computer system 1200.

Computer system 1200 can be one of various types, including a handheld portable device (e.g., an iPhone® cellular phone, an iPad® computing tablet, a personal digital assistant (PDA)), a wearable device (e.g., a Google Glass® head mounted display), a personal computer, a workstation, a mainframe, a kiosk, a server rack, or any other data processing system. Due to the ever-changing nature of computers and networks, the description of computer system 1200 depicted in FIG. 12 is intended only as a specific example. Many other configurations having more or fewer components than the system depicted in FIG. 12 are possible. Based on the disclosure and teachings provided herein, a person of ordinary skill in art can appreciate other ways and/or methods to implement the various aspects.

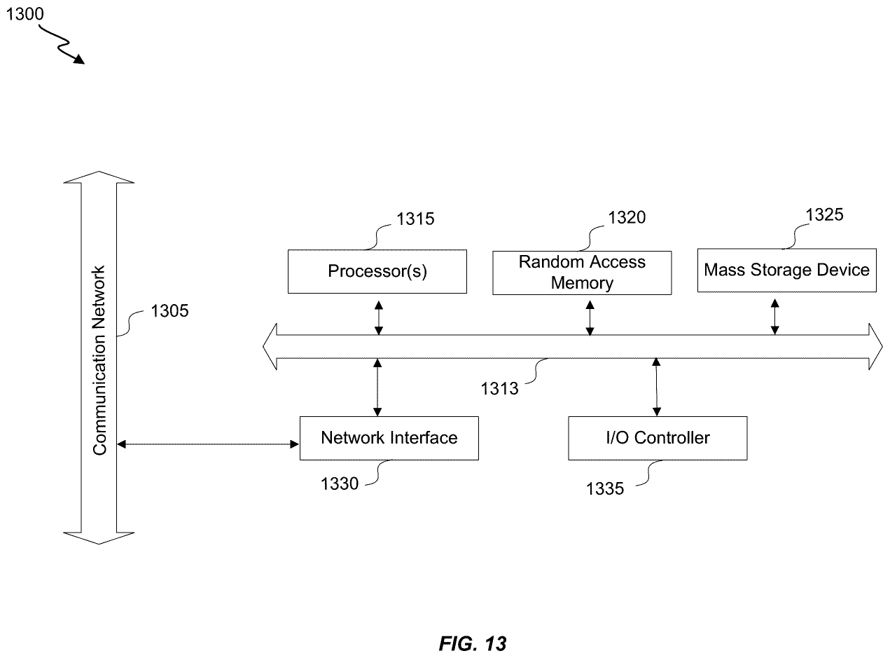

FIG. 13 illustrates an exemplary server which may include a processor, a memory, and a mass storage device. An exemplary Server 1300 may include a processor 1315, a memory (e.g., RAM) 1320, a bus 1310 which couples processor 1315 and memory 1320, a mass storage device 1325 (e.g., a magnetic or optical disk) coupled to processor 1315 and memory 1320 through an I/O controller 1335, and a network interface 1330 coupled to the processor and the memory. Network interface 1330 is further connected to a communication network 1305. Servers may be clustered together to handle more subject traffic and may include separate servers for different functions such as a database server, an application server, and a Web presentation server. Such servers may further include one or more mass storage devices 1325 such as a disk farm or a redundant array of independent disk (“RAID”) system for additional storage and data integrity. Read-only devices, such as compact disk drives and digital versatile disk drives, may also be connected to the servers. Suitable servers and mass storage devices are manufactured by, for example, Compaq, IBM, and Sun Microsystems. Generally, a server may operate as a source of content and provide any associated back-end processing, while an end user can be consumer of content provided by the server. However, it should be appreciated that many of the devices described above may be configured to respond to remote requests, thus operating as a server, and the devices described as servers may operate as end users of remote data sources. In contemporary peer-to-peer networks and environments such as RSS environments, the distinction between end users and servers is a blur. Accordingly, as used herein, the term “server” as used herein is generally intended to refer to any of the above-described servers, or any other device that may be used to provide content such as RSS feeds in a networked environment.

### Example 1

The purpose of this example is to showcase one of the example embodiments that includes training the machine-learning models in accordance with the process represented in FIG. 2, and features shown in Table 1. In one of the embodiments, several machine-learning models were implemented to predict resource usage. Various machine-learning models such as Random Forest model, AdaBoost model, Logistic Regression, Gradient boost and Xgboost machine model were trained using the relevant features identified in Table 1. 70 percent of the data was used for training, and 30 percent was used as test data. The training data included two years of data about subject characteristics and medical-care appointment/visit dates.

When the Adaboost model was used, the mean (median) root-mean square error was less than 0.3, and the mean absolute percentage error was less than 5%. The error was higher when any of the following models such as Random Forest, Logistic Regression, Gradient boost, and Xgboost were used.

Some embodiments of the present disclosure include a system including one or more data processors. In some embodiments, the system includes a non-transitory computer readable storage medium containing instruction which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, although the present invention as claimed has been specifically disclosed by embodiments and optional features, modification and variation of the concepts herein disclosed may be resorted to by those skilled in the art, and that such modifications and variations are within the scope of this invention as defined by the appended claims.

The present description provides preferred exemplary embodiments only, and is not intended to limit the scope, applicability, or configuration of the disclosure. Rather, the present description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the present description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

What is claimed is:

1. A computer-implemented method comprising:
receiving an input dataset from one or more data sources for a subject of a set of subjects;
detecting an outreach communication that has occurred or is scheduled to occur for the subject, wherein the outreach communication includes a recommendation that the subject seeks medical care at a particular medical facility;
extracting a set of features from the input dataset;
generating a derived feature from one or more features of the set of features;
predicting a likelihood of the subject seeking care at the particular medical facility within a predefined time period by processing the extracted set of features and derived features using a machine-learning model;
determining an upcoming resource demand at the particular medical facility based on the predicted likelihoods of the subjects seeking care at the particular medical facility;
detecting that the predicted upcoming resource demand exceeds a threshold; and
generating an output with a recommended action related to the particular medical facility in response to detecting that the predicted upcoming resource demand exceeds the threshold.

2. The computer-implemented method of claim 1, wherein a feature of the set of features indicates a prediction that critical medical care is sought by the subject.

3. The computer-implemented method of claim 1, wherein a feature of the set of features indicates an estimated total number of emergency-room visits that the subject has had within a defined time period or across a life of the subject.

4. The computer-implemented method of claim 1, wherein a feature of the set of features indicates whether the subject has one or more preconditions, one or more comorbidities, a count or statistic as to a number of times that the subject has been admitted into a hospital or a statistic characterizing a length of stay of one or more hospital admissions.

5. The computer-implemented method of claim 1, wherein the machine-learning model includes an Adaboost model, an ensemble model, self-learning model or one or more classifier sub-models.

6. The computer-implemented method of claim 1, wherein the threshold is specifically identified for the particular medical facility based on current resource allocations and/or scheduled resource allocations.

7. The computer-implemented method of claim 1, further comprising, in response to detecting that the predicted upcoming resource demand exceeds the threshold:
generating a proposed updated schedule that assigns resources to time slots associated with the particular medical facility, wherein the proposed updated schedule proposes adding a new resource to one or more time slots associated with the particular medical facility or proposes extending at least one time slot currently assigned to a given resource;
wherein the recommended action is to authorize and implement the proposed updated schedule.

8. The computer-implemented method of claim 1, wherein the recommended action is to adjust a subsequent outreach communication from recommending that another subject seek medical care at the particular medical facility to recommending that other subject seek medical care at a different medical facility.

9. The computer-implemented method of claim 1, wherein the particular medical facility is a first department in a hospital, and the recommended action is to reassign at least one resource from a second department in the hospital to the first department for a specified time period.

10. A system comprising:
one or more data processors; and
a non-transitory computer readable storage medium containing instructions which, when executed on the one or more data processors, cause the one or more data processors to perform actions including:
receiving an input dataset from one or more data sources for a subject of a set of subjects;
detecting an outreach communication that has occurred or is scheduled to occur for the subject, wherein the outreach communication includes a recommendation that the subject seeks medical care at a particular medical facility;
extracting a set of features from the input dataset;
generating a derived feature from one or more features of the set of features;
predicting a likelihood of the subject seeking care at the particular medical facility within a predefined time period by processing the extracted set of features and derived features using a machine-learning model;
determining an upcoming resource demand at the particular medical facility based on the predicted likelihoods of the subjects seeking care at the particular medical facility;
detecting that the predicted upcoming resource demand exceeds a threshold; and
generating an output with a recommended action related to the particular medical facility in response to detecting that the predicted upcoming resource demand exceeds the threshold.

11. The system of claim 10, wherein a feature of the set of features indicates a prediction that critical medical care is sought by the subject.

12. The system of claim 10, wherein a feature of the set of features indicates an estimated total number of emergency-room visits that the subject has had within a defined time period or across a life of the subject.

13. The system of claim 10, wherein a feature of the set of features indicates whether the subject has one or more preconditions, one or more comorbidities, a count or statistic as to a number of times that the subject has been admitted into a hospital or a statistic characterizing a length of stay of one or more hospital admissions.

14. The system of claim 10, wherein the machine-learning model includes an Adaboost model, an ensemble model, self-learning model or one or more classifier sub-models.

15. The system of claim 10, further comprising, in response to detecting that the predicted upcoming resource demand exceeds the threshold:
generating a proposed updated schedule that assigns resources to time slots associated with the particular medical facility, wherein the proposed updated schedule proposes adding a new resource to one or more time slots associated with the particular medical facility or proposes extending at least one time slot currently assigned to a given resource;
wherein the recommended action is to authorize and implement the proposed updated schedule.

16. A computer-program product tangibly embodied in a non-transitory machine-readable storage medium, including instructions configured to cause one or more data processors to perform action including:
receiving an input dataset from one or more data sources for a subject of a set of subjects;
detecting an outreach communication that has occurred or is scheduled to occur for the subject, wherein the outreach communication includes a recommendation that the subject seeks medical care at a particular medical facility;
extracting a set of features from the input dataset;
generating a derived feature from one or more features of the set of features;
predicting a likelihood of the subject seeking care at the particular medical facility within a predefined time period by processing the extracted set of features and derived features using a machine-learning model;
determining an upcoming resource demand at the particular medical facility based on the predicted likelihoods of the subjects seeking care at the particular medical facility;
detecting that the predicted upcoming resource demand exceeds a threshold; and
generating an output with a recommended action related to the particular medical facility in response to detecting that the predicted upcoming resource demand exceeds the threshold.

17. The computer-program product of claim 16, wherein a feature of the set of features indicates a prediction that critical medical care is sought by the subject.

18. The computer-program product of claim 16, wherein a feature of the set of features indicates an estimated total number of emergency-room visits that the subject has had within a defined time period or across a life of the subject.

19. The computer-program product of claim 16, wherein the machine-learning model includes an Adaboost model, an ensemble model, self-learning model or one or more classifier sub-models.

20. The computer-program product of claim 16, further comprising, in response to detecting that the predicted upcoming resource demand exceeds the threshold:
generating a proposed updated schedule that assigns resources to time slots associated with the particular medical facility, wherein the proposed updated schedule proposes adding a new resource to one or more time slots associated with the particular medical facility or proposes extending at least one time slot currently assigned to a given resource;
wherein the recommended action is to authorize and implement the proposed updated schedule.
